SEQUENCE LISTING (33 sequences)

SEQ ID NO: 1 - DNA, 27 bp, Human immunodeficiency virus 1
ttacaccctgtgagcctgcatgggatg

SEQ ID NO: 2 - DNA, 27 bp, Human immunodeficiency virus 1
ttagagtggaggtttgacagccgccta

SEQ ID NO: 3 - DNA, 27 bp, Human immunodeficiency virus 1
tttggatggtgctacaagctagtacca

SEQ ID NO: 4 - DNA, 27 bp, Human immunodeficiency virus 1
tttgacagccgcctagcatttcatcac

SEQ ID NO: 5 - DNA, 27 bp, Human immunodeficiency virus 1
tttcatcacatggcccgagagctgcat

SEQ ID NO: 6 - DNA, 27 bp, Human immunodeficiency virus 1
tttccgctggggactttccagggaggc

SEQ ID NO: 7 - DNA, 27 bp, Human immunodeficiency virus 1
tttccagggaggcgtggcctgggcggg

SEQ ID NO: 8 - DNA, 27 bp, Human immunodeficiency virus 1
tttttgcttgtactgggtctctctggt

SEQ ID NO: 9 - DNA, 27 bp, Human immunodeficiency virus 1
ttttgcttgtactgggtctctctggtt

SEQ ID NO: 10 - DNA, 27 bp, Human immunodeficiency virus 1
tttgcttgtactgggtctctctggtta

SEQ ID NO: 11 - DNA, 27 bp, Human immunodeficiency virus 1
ttcactcccaacgaagacaagatatcc

SEQ ID NO: 12 - DNA, 27 bp, Human immunodeficiency virus 1
ttccctgattggcagaactacacacca

SEQ ID NO: 13 - DNA, 27 bp, Human immunodeficiency virus 1
ttcatcacatggcccgagagctgcatc

SEQ ID NO: 14 - DNA, 27 bp, Human immunodeficiency virus 1
ttcaagaactgctgacatcgagcttgc

SEQ ID NO: 15 - DNA, 27 bp, Human immunodeficiency virus 1
ttccgctggggactttccagggaggcg

SEQ ID NO: 16 - DNA, 27 bp, Human immunodeficiency virus 1
ttccagggaggcgtggcctgggcggga

SEQ ID NO: 17 - DNA, 27 bp, Human immunodeficiency virus 1
ttgatctgtggatctaccacacacaag

SEQ ID NO: 18 - DNA, 27 bp, Human immunodeficiency virus 1
ttggcagaactacacaccagggccagg

SEQ ID NO: 19 - DNA, 27 bp, Human immunodeficiency virus 1
ttggatggtgctacaagctagtaccag

SEQ ID NO: 20 - DNA, 27 bp, Human immunodeficiency virus 1
ttgagcaagagaaggtagaagaagcca

SEQ ID NO: 21 - DNA, 27 bp, Human immunodeficiency virus 1
ttgttacaccctgtgagcctgcatggg

SEQ ID NO: 22 - DNA, 27 bp, Human immunodeficiency virus 1
ttgctacaagggactttccgctgggga

SEQ ID NO: 23 - DNA, 27 bp, Human immunodeficiency virus 1
ttgcttgtactgggtctctctggttag

SEQ ID NO: 24 - DNA, 27 bp, Human immunodeficiency virus 1
tggtactgggtctctctggttagacca

SEQ ID NO: 25 - DNA, 27 bp, Human immunodeficiency virus 1
ttagaccagatctgagcctgggagctc

SEQ ID NO: 26 - DNA, 27 bp, Human immunodeficiency virus 1
ttaagcctcaataaagcttgccttgag

SEQ ID NO: 27 - DNA, 27 bp, Human immunodeficiency virus 1
ttgccttgagtgcttcaagtagtgtgt

SEQ ID NO: 28 - DNA, 27 bp, Human immunodeficiency virus 1
ttgagtgcttcaagtagtgtgtgcccg

SEQ ID NO: 29 - DNA, 26 bp, Human immunodeficiency virus 1
ttagtcagtgtggaaaatctctagca

SEQ ID NO: 30 - DNA, 27 bp, Human immunodeficiency virus 1
ttcaagtagtgtgtgcccgtctgttgt

SEQ ID NO: 31 - DNA, 27 bp, Human immunodeficiency virus 1
ttttagtcagtgtggaaaatctctagc

SEQ ID NO: 32 - DNA, 27 bp, Human immunodeficiency virus 1
tttagtcagtgtggaaaatctctagca

SEQ ID NO: 33 - DNA, 27 bp, Human immunodeficiency virus 1
ttgagtgcttcaagtagtgtgtgcccg

(19) United States
(12) Patent Application Publication
(10) Pub. No.: US 2019/0083656 A1
(43) Pub. Date: Mar. 21, 2019

(54) METHODS AND COMPOSITIONS UTILIZING CPF1 FOR RNA-GUIDED GENE EDITING

(71) Applicant: Temple University - of the Commonwealth System of Higher Education, Philadelphia, PA (US)

(72) Inventors: Kamel Khalili, Bala Cynwyd, PA (US); Thomas Malcolm, Bedminster, NJ (US)

(21) Appl. No.: 15/768,241

(22) PCT Filed: Oct. 14, 2016
(86) PCT No.: PCT/US16/57069

§ 371 Date: Apr. 13, 2018

Related U.S. Application Data
(60) Provisional application No. 62/242,772, filed on Oct. 16, 2015.

(51) Int. Cl. A61K 48/00; A61P 31/18

(52) U.S. Cl. CPC A61K 48/0066; A61P 31/18; A61K 48/0091; A61K 48/0083; A61K 48/0041

(57) ABSTRACT

Compositions include endonucleases of the family Cpf1 (CRISPR from Prevotella and Francisella 1); and at least one guide RNA (gRNA) complementary to a target sequence in a gene to specifically guide the Cpf1 endonuclease to the target site in a host cell in vitro or in vivo. Methods of treating a subject include the use of one or more of these compositions.

### STATEMENT REGARDING FEDERALLY SPONSORED RESEARCH

This invention was made with U.S. government support under grant numbers R01MH093271, R01NS087971, and P30MH092177 awarded by the National Institutes of Health. The U.S. government may have certain rights in the invention.

### FIELD OF THE INVENTION

The present invention relates to compositions and methods for cleavage of DNA at specific target sites, to enable gene editing. The compositions include endonucleases of the family Cpf1 (CRISPR from Prevotella and Francisella 1); and at least one guide RNA (gRNA) complementary to a target sequence in a DNA, to guide the Cpf1 endonuclease to a target site, such as a site associated with a HIV-1 proviral DNA integrated in a host cell genome.

### BACKGROUND

Great progress has been made in the field of gene editing. Improved, more accurate, and simpler methods for gene editing have been introduced during the last decade. A major innovation was the introduction of the gene editing system referred to as CRISPR/Cas9 (CRISPR, clustered regularly interspersed short palindromic repeats; Cas9, CRISPR associated protein 9). CRISPR/Cas9 systems were originally discovered as antiviral defense mechanisms used by certain bacterial species, to recognize and cleave characteristic DNA sequences of bacteriophage viruses. CRISPR/Cas9 systems have been manipulated to carry out gene editing functions in a broad range of organisms including yeast, Drosophila, zebrafish, C. elegans, and mice, and has been heavily used by several laboratories in a broad range of in vivo and in vitro studies toward human diseases (Di Carlo J. E. et al., Nucl Acids Res 41:4336-4346 (2013); Gratz S. J. et al., Genetics 194, 1029-1035 (2013); Hwang W. Y. et al., Nature Biotech 31, 227-229, (2013); Yu L. et al., OncoTargets Ther 8, 37-44 (2015); Hu W. et al., Proc Natl Acad Sci USA 111, 11461-11466 (2014)).

In a CRISPR/Cas9 system, gene editing complexes are assembled. Each complex includes a Cas9 nuclease and a guide RNA (gRNA) complementary to a target sequence in a targeted DNA. The gRNA directs the Cas9 nuclease to engage and cleave the targeted DNA at or near the target sequence. The cleavage produces a blunt double stranded break that, without further intervention, triggers repair enzymes to rejoin or replace DNA sequences at or near the cleavage site. These repairs are usually defective, resulting in one or more mutations into the target DNA, such as nucleotide substitutions, insertions, and deletions. The mutations can included the excision of long stretches of DNA, especially when multiple target sites are cleaved simultaneously. If copies of desired stretches of DNA are introduced during the editing process, they can be spliced into the cleavage site by a process known as homology directed repair (HDR) (Sander J. D. and Joung L. K., Nature Biotech 32, 347-355 (2014)).

Recently, CRISPR/Cas9 systems have been modified to enable the recognition and cleavage of target sequences of retroviruses integrated into the human genome. HIV-1 proviral genomes are a primary target, since their integration into host T cells represents a latent infection that can be activated to trigger AIDS symptoms. gRNAs have been developed to recognize DNA sequences positioned within HIV-1 long terminal repeat (LTR) sequences. Through the use of these RNAs in the CRISPR/Cas9 system, integrated HIV-1 sequences were inactivated, and in most cases completely eradicated, in human T cells, microglial cells, and monocytic cells. The most effective excisions were produced when at least two gRNAs were employed, with each targeting a different site in the LTR. Stable expression of the CRISPR/Cas9 components conferred resistance to further infection in the human T cells (Hu W. et al., Proc Natl Acad Sci USA 111, 11461-11466 (2014); Khalili et al., 2015, International Patent Application No. WO2015/031775 to Khalili, et al.). CRISPR/Cas systems have also been developed for the inactivation of human neurotropic JC virus (JCV). This human polyoma virus infects cells of the nervous system, causing a fatal demyelinating disease (Wollebo H. S. et al., Ann. Neurol. 77:560-570 (2015)).

CRISPR/Cas9 systems have many additional uses. For example, at least one human genetic disease has been cured in an animal model through use of CRISPR/Cas9. Hereditary tyrosinemia type I (HTI), is a fatal genetic disease caused by a point mutation of fumarylacetoacetate hydrolase (FAH), an enzyme essential for protein metabolism. A point mutation of a single NT causes the disease. gRNAs were designed to cause a double stranded break adjacent to a stretch of DNA containing the mutation. The gRNAs and Cas9 were injected into the mice, along with a 199 nucleotide single stranded donor DNA encoding the same stretch the normal FAH gene. The donor DNA was successfully spliced in to replace the mutant DNA, by the process of homology directed repair (HDR). The liver cells of the mice regained their normal function, and the mice were cured of HTI (Yin H. et al., Nature Biotech 32, 551-554 (2014)).

In another example, a CRISPR/Cas9 system can be adapted to attach detectable labels, such as fluorescent labels, to specific target sites in a genome. This application is useful in fluorescent imaging of target sites such as HIV proviral incorporation sites, and retrotransposons. The application is also useful to mark multiple DNA motifs for whole genome sequencing techniques, such as those employing the IRYS® single-molecule DNA mapping system. In these labelling systems, a catalytically deficient Cas9 is employed. The catalytically deficient Cas9 is capable of forming a complex with a gRNA, and binding to a target site, but not of cleaving the DNA at that site. The catalytically deficient Cas9 is labelled, often with a fluorescent protein such as extended green fluorescent protein (EGFP) and/or red fluorescent protein (RFP). The Cas9/gRNA complex localizes to the target site, tagging that site with the fluorescent label.

Unfortunately, the CRISPR/Cas9 system has drawbacks that limit its potential. Many of these drawbacks are inherent in the S. pyogenes Cas9 endonuclease that is employed in most of the systems. This Cas9 endonuclease can only attach to and cleave target DNA sequences that are adjacent to short naturally occurring sequences called PAMs (protospacer adjacent motifs). The PAMs generally include the trinucleotide NGG. Therefore, the use of CRISPR/Cas9 systems is limited to target sequences adjacent to NGG PAMs.

Furthermore, Cas9 cleaves at the same site in both strands of double stranded DNA, creating a break with “blunt ends”. Blunt ends are not favorable to precise insertion of desired DNA sequences at the cleavage site. Staggered cuts, leaving an “overhang” in each strand are preferred.

Another disadvantage of Cas9 is its relatively large size, which makes it difficult to insinuate into the nucleus of a living cell. Finally, CRISPR/Cas9 systems, while much simpler and easier to use than systems of the prior art, are still complex. The gRNA is actually composed of a duplex of two smaller RNAs: a mature CRISPR RNA (crRNA), and a trans-activated small RNA (tracrRNA).

There is a great need for gene editing systems that expand the repertoire of potential DNA target sites. In particular, there is a need for gene editing systems that are more favorable for the insertion of desired genes, and provides a smaller, simpler gene editing complex, than do CRISPR/Cas9 systems.

### SUMMARY

The present invention provides compositions for use in inactivating target genes in the genome of a host cell. The compositions include isolated nucleic acid sequences encoding a Cpf1 (CRISPR from Prevotella and Francisella 1) endonuclease, and at least one guide RNA (gRNA), which is complementary to a target DNA sequence in the target gene. The gRNA directs the Cpf1 endonuclease to the target DNA sequence. The resulting double stranded breaks in the DNA inactivate the target gene by causing point mutations, insertions, deletions, or the complete excision of a stretch of DNA including the target gene. The present invention also provides methods of using these compositions to inactivate target genes.

The present invention further provides compositions for use in inactivating integrated viral DNA in the genome of a host cell. The compositions include isolated nucleic acid sequences encoding a Cpf1 endonuclease, and at least one guide gRNA, which is complementary to a target DNA sequence in the proviral DNA. In some embodiments, the target sequence is in the long terminal repeat (LTR) of HIV, for example, HIV-1 proviral DNA. The present invention still further provides methods of using these compositions to inactivate integrated proviruses.

The present invention also provides expression vectors for use in inactivating a target gene the genome of a host cell. The vectors induce the expression of at least one Cpf1 endonuclease, and at least one gRNA complementary to a target sequence in the target gene. A preferred vector is a lentiviral expression vector.

The present invention further provides methods of preventing a viral infection of host cells of a patient at risk of viral infection. The method includes establishing, in the host cells, the stable expression of a Cpf1 endonuclease and at least one gRNA, which is complementary to a target sequence in the viral genome. An exemplary viral infection is HIV-1 infection.

The present invention still further provides pharmaceutical compositions for inactivating a provirus in the cells of a mammalian subject. The compositions include nucleic acid sequences encoding a Cpf1 endonuclease, and at least one gRNA that is complementary to a target sequence in a proviral DNA. The isolated nucleic acid sequences are included in at least one expression vector. The present invention also provides methods of using these pharmaceutical compositions to inactivate a provirus in the host cells.

The present invention further provides methods for correcting a genetic disease in a cell. The methods can be applied to any cell whose DNA includes a disease-causing mutated DNA sequence. In these methods, the cell is exposed to at least one gRNA that is complementary to a target site adjacent to the disease-causing mutated DNA sequence. The gRNA directs a Cpf1 endonuclease to cause a double stranded break adjacent to the target site. The cell is then exposed to a single stranded donor oligonucleotide including a wild type DNA sequence corresponding to the disease-causing mutated DNA sequence. The mutated DNA sequence is replaced with the wild type DNA sequence, and the genetic disease is corrected.

The present invention still further provides methods for detecting specific DNA sequences with a detectable label, such as a fluorescent label, for the purposes of diagnosis and genomic analysis. The methods include the nicking of a target site in DNA with a Cpf1 mutant with nickase activity, and the incorporation of labelled nucleotides at the nicked site.

The present invention also provides compositions for detecting specific DNA sequences, for the purposes of diagnosis and genomic analysis. The compositions include a catalytically deficient Cpf1, which can be directed to a specific DNA sequence by a gRNA, but which cannot cleave the DNA at the sequence. The catalytically deficient Cpf1 is labelled, for example with a fluorescent tag, resulting in the labelling of the specific DNA sequence.

All compositions of the present invention that include a CRISPR/Cpf1 system can of course be combined with a CRISPR/Cas9 system, to obtain the benefits of both the Cpf1 and Cas9 endonucleases.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

The details of one or more embodiments of the invention are set forth in the accompanying drawings and the description below. Other features, objects, and advantages of the invention will be apparent from the description and drawings, and from the claims.

FIG. 1 shows a diagram of a Cpf1/gRNA complex acting upon a target DNA sequence.

### DETAILED DESCRIPTION

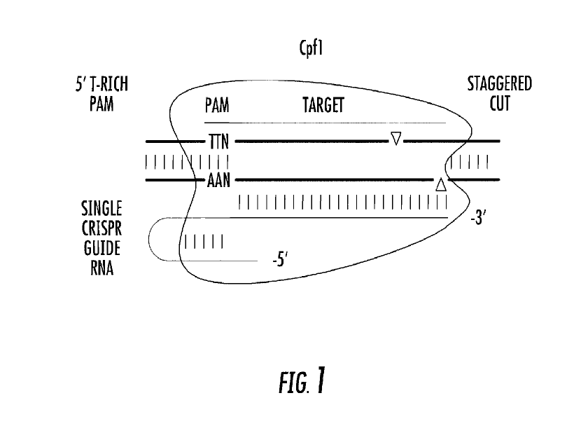

The present invention is based, in part, on the discovery bacterial CRISPR systems that utilize an endonuclease other than Cas9, and that some of these nucleases can serve as alternatives, sometimes superior alternatives, to Cas9 in CRISPR systems. Of special potential are certain members of the endonuclease family Cpf1 (CRISPR from Prevotella and Francisella 1) (Zetsche, et al., 2015). Two Cpf1 endonucleases have so far been shown to be effective at editing genes in a cultured human kidney cell system: Acidaminococcus sp. BV3L6 Cpf1, and Lachnospiraceae bacterium ND2006 Cpf1. A schematic diagram of a gRNA/Cpf1 complex is shown in FIG. 1.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

All genes, gene names, and gene products disclosed herein are intended to correspond to homologs from any species for which the compositions and methods disclosed herein are applicable. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, are intended to encompass homologous and/or orthologous genes and gene products from other species.

The articles “a” and “an” are used herein to refer to one or to more than one (i.e., to at least one) of the grammatical object of the article. By way of example, “an element” means one element or more than one element. Thus, recitation of “a cell”, for example, includes a plurality of the cells of the same type. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

“About” as used herein when referring to a measurable value such as an amount, a temporal duration, and the like, is meant to encompass variations of +/−20%, +/−10%, +/−5%, +/−1%, or +/−0.1% from the specified value, as such variations are appropriate to perform the disclosed methods. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

The term “anti-viral agent” as used herein, refers to any molecule that is used for the treatment of a virus and include agents which alleviate any symptoms associated with the virus, for example, anti-pyretic agents, anti-inflammatory agents, chemotherapeutic agents, an anti-pyretic agent, anti-inflammatory agent, anti-fungal agent, anti-parasitic agent, chemotherapeutic agent, antibiotics, immunomodulating agent, and the like. An antiviral agent includes, without limitation: antibodies, aptamers, adjuvants, anti-sense oligonucleotides, chemokines, cytokines, immune stimulating agents, immune modulating agents, B-cell modulators, T-cell modulators, NK cell modulators, antigen presenting cell modulators, enzymes, siRNA's, ribavirin, ribozymes, protease inhibitors, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, or combinations thereof. An immunomodulating agent comprises but is not limited to cytokines, lymphokines, T cell co-stimulatory ligands, chemokines, adjuvants, etc.

The term “antibody” as used herein comprises one or more virus specific binding domains which bind to and aid in the immune mediated-destruction and clearance of the virus, e.g. HIV. The antibody or fragments thereof, comprise IgA, IgM, IgG, IgE, IgD or combinations thereof.

The term “eradication” of a virus, e.g. HIV, as used herein, means that that virus is unable to replicate, the genome is deleted, fragmented, degraded, genetically inactivated, or any other physical, biological, chemical or structural manifestation, that prevents the virus from being transmissible or infecting any other cell or subject resulting in the clearance of the virus in vivo. In some cases, fragments of the viral genome may be detectable, however, the virus is incapable of replication, or infection etc.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “exogenous” indicates that the nucleic acid or polypeptide is part of, or encoded by, a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The term “immunoregulatory” or “immune cell modulator” or “immunomodulating agent” is meant a compound, composition or substance that is immunogenic (i.e. stimulates or increases an immune response) or immunosuppressive (i.e. reduces or suppresses an immune response). “Cells of the immune system” or “immune cells”, is meant to include any cells of the immune system that may be assayed or involved in mounting an immune response, including, but not limited to, B lymphocytes, also called B cells, T lymphocytes, also called T cells, natural killer (NK) cells, natural killer T (NK) cells, lymphokine-activated killer (LAK) cells, monocytes, macrophages, neutrophils, granulocytes, mast cells, platelets, Langerhans cells, stem cells, dendritic cells, peripheral blood mononuclear cells, tumor-infiltrating (TIL) cells, gene modified immune cells including hybridomas, drug modified immune cells, and derivatives, precursors or progenitors of the above cell types. The functions or responses to an antigen can be measured by any type of assay, e.g. RIA, ELISA, FACS, Western blotting, etc.

The term “induces or enhances an immune response” is meant causing a statistically measurable induction or increase in an immune response over a control sample to which the peptide, polypeptide or protein has not been administered. Conversely, “suppression” of an immune response is a measurable decrease in an immune response over a control sample to which the peptide, polypeptide or protein has been administered, for example, as in the case of suppression of an immune response in an auto-immune scenario. Preferably the induction or enhancement of the immune response results in a prophylactic or therapeutic response in a subject. Examples of immune responses are increased production of type I IFN, increased resistance to viral and other types of infection by alternate pathogens. The enhancement of immune responses to viruses (anti-virus responses), or the development of vaccines to prevent virus infections or eliminate existing viruses.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes: a recombinant DNA which is part of a hybrid gene encoding additional polypeptide sequence, complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like.

The nucleic acid sequences may be “chimeric,” that is, composed of different regions. In the context of this invention “chimeric” compounds are oligonucleotides, which contain two or more chemical regions, for example, DNA region(s), RNA region(s), PNA region(s) etc. Each chemical region is made up of at least one monomer unit, i.e., a nucleotide. These sequences typically comprise at least one region wherein the sequence is modified in order to exhibit one or more desired properties.

The term “target nucleic acid” sequence refers to a nucleic acid (often derived from a biological sample), to which the oligonucleotide is designed to specifically hybridize. The target nucleic acid has a sequence that is complementary to the nucleic acid sequence of the corresponding oligonucleotide directed to the target. The term target nucleic acid may refer to the specific subsequence of a larger nucleic acid to which the oligonucleotide is directed or to the overall sequence (e.g., gene or mRNA). The difference in usage will be apparent from context.

In the context of the present invention, the following abbreviations for the commonly occurring nucleic acid bases are used, “A” refers to adenosine, “C” refers to cytosine, “G” refers to guanosine, “T” refers to thymidine, and “U” refers to uridine.

Unless otherwise specified, a “nucleotide sequence encoding” an amino acid sequence includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g., subcutaneous (s.c.), intravenous (i.v.), intramuscular (i.m.), or intrasternal injection, or infusion techniques.

The terms “patient” or “individual” or “subject” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters, and primates.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “percent sequence identity” or having “a sequence identity” refers to the degree of identity between any given query sequence and a subject sequence.

The terms “pharmaceutically acceptable” (or “pharmacologically acceptable”) refer to molecular entities and compositions that do not produce an adverse, allergic or other untoward reaction when administered to an animal or a human, as appropriate. The term “pharmaceutically acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance.

The term “polynucleotide” is a chain of nucleotides, also known as a “nucleic acid”. As used herein polynucleotides include, but are not limited to, all nucleic acid sequences which are obtained by any means available in the art, and include both naturally occurring and synthetic nucleic acids.

The term “transfected” or “transformed” or “transduced” means to a process by which exogenous nucleic acid is transferred or introduced into the host cell. A “transfected” or “transformed” or “transduced” cell is one which has been transfected, transformed or transduced with exogenous nucleic acid. The transfected/transformed/transduced cell includes the primary subject cell and its progeny.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Examples of vectors include but are not limited to, linear polynucleotides, polynucleotides associated with ionic or amphiphilic compounds, plasmids, and viruses. Thus, the term “vector” includes an autonomously replicating plasmid or a virus. The term is also construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Where any amino acid sequence is specifically referred to by a Swiss Prot. or GENBANK Accession number, the sequence is incorporated herein by reference. Information associated with the accession number, such as identification of signal peptide, extracellular domain, transmembrane domain, promoter sequence and translation start, is also incorporated herein in its entirety by reference.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Compositions for Eradication of Retrovirus in Cells or Subjects

Embodiments of the invention are directed to compositions comprising an endonuclease and at least one guide RNA (gRNA) sequence, the guide RNA being complementary to a target nucleic acid sequence in a target gene. In some embodiments, the compositions disclosed herein include nucleic acids encoding an endonuclease, such as Cas9. In certain embodiments, the compositions include isolated nucleic acid sequences encoding a Cpf1 (CRISPR from Prevotella and Francisella 1) endonuclease, and at least one guide RNA (gRNA), which is complementary to a target DNA sequence in the target gene. The gRNA directs the Cpf1 endonuclease to the target DNA sequence. The resulting double stranded breaks in the DNA inactivate the target gene by causing point mutations, insertions, deletions, or the complete excision of a stretch of DNA including the target gene.

In other embodiments, nuclease systems that can be used include, without limitation, zinc finger nucleases, transcription activator-like effector nucleases (TALENs), meganucleases, or any other system that can be used to degrade or interfere with viral nucleic acid without interfering with the regular function of the host's genetic material.

The present invention also provides methods of using these compositions to inactivate target genes and eradicate a virus infection in a host. The methods of the invention may be used to remove viral or other foreign genetic material from a host organism, without interfering with the integrity of the host's genetic material. A nuclease may be used to target viral nucleic acid, thereby interfering with viral replication or transcription or even excising the viral genetic material from the host genome. The nuclease may be specifically targeted to remove only the viral nucleic acid without acting on host material either when the viral nucleic acid exists as a particle within the cell or when it is integrated into the host genome. The compositions may be used to target viral nucleic acid in any form or at any stage in the viral life cycle. The targeted viral nucleic acid may be present in the host cell as independent particles. In a preferred embodiment, the viral infection is latent and the viral nucleic acid is integrated into the host genome. Any suitable viral nucleic acid may be targeted for cleavage and digestion.

Gene Editing Agents:

Compositions of the invention include at least one gene editing agent, comprising CRISPR-associated nucleases such as Cas9 and Cpf1 gRNAs, Argonaute family of endonucleases, clustered regularly interspaced short palindromic repeat (CRISPR) nucleases, zinc-finger nucleases (ZFNs), transcription activator-like effector nucleases (TALENs), meganucleases, other endo- or exo-nucleases, or combinations thereof. See Schiffer, 2012, J Virol 88(17):8920-8936, incorporated by reference.

As referenced above, Argonaute is another potential gene editing system. Argonautes are a family of endonucleases that use 5′ phosphorylated short single-stranded nucleic acids as guides to cleave targets (Swarts, D. C. et al. The evolutionary journey of Argonaute proteins. Nat. Struct. Mol. Biol. 21, 743-753 (2014)). Similar to Cas9, Argonautes have key roles in gene expression repression and defense against foreign nucleic acids (Swarts, D. C. et al. Nat. Struct. Mol. Biol. 21, 743-753 (2014); Makarova, K. S., et al. Biol. Direct 4, 29 (2009). Molloy, S. Nat. Rev. Microbiol. 11, 743 (2013); Vogel, J. Science 344, 972-973 (2014). Swarts, D. C. et al. Nature 507, 258-261 (2014); Olovnikov, I., et al. Mol. Cell 51, 594-605 (2013)). However, Argonautes differ from Cas9 in many ways Swarts, D. C. et al. The evolutionary journey of Argonaute proteins. Nat. Struct. Mol. Biol. 21, 743-753 (2014)). Cas9 only exist in prokaryotes, whereas Argonautes are preserved through evolution and exist in virtually all organisms; although most Argonautes associate with single-stranded (ss)RNAs and have a central role in RNA silencing, some Argonautes bind ssDNAs and cleave target DNAs (Swarts, D. C. et al. Nature 507, 258-261 (2014); Swarts, D. C. et al. Nucleic Acids Res. 43, 5120-5129 (2015)). guide RNAs must have a 3′ RNA-RNA hybridization structure for correct Cas9 binding, whereas no specific consensus secondary structure of guides is required for Argonaute binding; whereas Cas9 can only cleave a target upstream of a PAM, there is no specific sequence on targets required for Argonaute. Once Argonaute and guides bind, they affect the physicochemical characteristics of each other and work as a whole with kinetic properties more typical of nucleic-acid-binding proteins (Salomon, W. E., et al. Cell 162, 84-95 (2015)).

The composition can also include C2c2—the first naturally-occurring CRISPR system that targets only RNA. The Class II type VI-A CRISPR-Cas effector “C2c2” demonstrates an RNA-guided RNase function. C2c2 from the bacterium Leptotrichia shahii provides interference against RNA phage. In vitro biochemical analysis show that C2c2 is guided by a single crRNA and can be programmed to cleave ssRNA targets carrying complementary protospacers. In bacteria, C2c2 can be programmed to knock down specific mRNAs. Cleavage is mediated by catalytic residues in the two conserved HEPN domains, mutations in which generate catalytically inactive RNA-binding proteins. These results demonstrate the capability of C2c2 as a new RNA-targeting tools. C2c2 can be programmed to cleave particular RNA sequences in bacterial cells. The RNA-focused action of C2c2 complements the CRISPR-Cas9 system, which targets DNA, the genomic blueprint for cellular identity and function. The ability to target only RNA, which helps carry out the genomic instructions, offers the ability to specifically manipulate RNA in a high-throughput manner—and manipulate gene function more broadly.

In some embodiments, one or more guide RNAs that are complementary to a target sequence of HIV may also be encoded. Accordingly, in some embodiments composition for use in inactivating a proviral DNA integrated into the genome of a host cell latently infected with human immunodeficiency virus (HIV), the composition comprises at least one isolated nucleic acid sequence encoding a Clustered Regularly Interspaced Short Palindromic Repeat (CRISPR)-associated endonuclease, and at least one guide RNA (gRNA), said at least one gRNA having a spacer sequence that is complementary to a target sequence in a long terminal repeat (LTR) of a proviral HIV DNA.

Cpf1 Endonucleases.

Cas9 is guided by a mature crRNA that contains about 20 base pairs (bp) of unique target sequence (called spacer) and a trans-activated small RNA (tracrRNA) that serves as a guide for ribonuclease III-aided processing of pre-crRNA. The crRNA:tracrRNA duplex directs Cas9 to target DNA via complementary base pairing between the spacer on the crRNA and the complementary target sequence (also called protospacer) on the target DNA. Cas9 recognizes a guanine rich trinucleotide (NGG) protospacer adjacent motif (PAM) to specify the cut site (the 3rd nucleotide from PAM). The PAM is adjacent to the 3′ end of the target sequence.

In contrast, Cpf1 recognizes a thymine rich PAM, with a consensus sequence TTN, and that PAM is located at the 5′ end of the target sequence. This gives a CRISPR/Cpf1 system a different repertoire of targets from a CRISPR/Cas9 system, expanding the spectrum of available gene editing targets.

Cpf1-Mediated Cleavage is Favorable for Gene Editing.

As previously stated, Cas9 makes a blunt ended cut in double stranded DNA. This promotes error prone repair and genetic inactivation, but is not favorable for splicing a desired segment of DNA into the cut site. In contrast, Cpf1 makes a staggered cut, leaving a five nucleotide overhang in each DNA strand. This is a favorable cut for incorporating a desired DNA segment, for example by homology-directed repair. Furthermore, the cut site is at the distal end of the target site, far from the region that is most important in determining target specificity, the “seed” sequence near the PAM. With the seed sequence left intact, multiple rounds of editing are possible.

Cpf1 Systems are Simpler and Smaller than Cas9 Systems.

In order to function, CRISPR/Cas9 system require the processing and assembly of two substituent RNAs, crRNA, which contains the spacer sequence, and tracrRNA. The crRNA and tracrRNA have been engineered into hybrid molecule known as a single small guide RNA (sgRNA), which provides a simpler but still large and complex system. In contrast, all binding and enzymatic functions of Cpf1 require only a single guide RNA, termed gRNA. This simplicity facilitates the design and use of CRISPR/Cpf1 systems.

Cpf1 also lacks one of the two nuclease domains found in Cas9. As a smaller molecule it should be easier to transport, for example, through nuclear pores, to target sites.

The advantages of CRISPR/Cpf1 systems are applied to a variety of purposes in the present invention.

CRISPR/Cpf1 Compositions.

The present invention encompasses compositions for inactivating a target gene in the genome of a host cell, including at least one Cpf1 endonuclease, and at least one gRNA), with the at least one gRNA being complementary to a target sequence in the target gene. When a gRNA is described as being complementary to a target DNA sequence, it will be understood that it is the spacer sequence of the gRNA that is actually complementary to the target DNA sequence.

The preferred embodiments of Cpf1 are those from Acidaminococcus sp. BV3L6 Cpf1, and Lachnospiraceae bacterium ND2006. These Cpf1 family members have thoroughly characterized, and have been shown to be approximately as effective as Cas9 in editing the DNMT1 gene in human kidney cells (Zetsche B. et al., Cell 163, 1-13 Oct. 22, 2015).

The sequences of gRNAs of the present invention will depend on the sequence of specific target sites selected for editing. In general, the gRNAs are predicted to be complementary to target DNA sequences that are immediately 3′ to a thymine rich PAM, of sequence 5′TTN. The gRNA sequence can be a sense or anti-sense sequence. The gRNA sequence may or may not include the complement to the PAM sequence. The gRNA sequence can include additional 5′ and/or 3′ sequences that may not be complementary to a target sequence. The gRNA sequence can have less than 100% complementarity to a target sequence, for example 95% complementarity. The gRNA nucleic acid sequences have a sequence complementary to a coding or a non-coding target sequence. The gRNA sequences can be employed in a multiplex configuration, including combinations of two, three, four, five, six, seven, eight, nine, ten, or more different gRNAs. It has been established in CRISPR/Cas9 systems that a duplex “two cut” strategy, employing two different gRNAs targeted to sites in the HIV-1 LTR can cause the excision of the entire stretch of DNA between the cleavage sites (Hu W. et al., Proc Natl Acad Sci USA 111, 11461-11466 (2014)). It is likely that a duplex gRNA configuration is also effective at producing excisions in the CRISPR/Cpf1 system, in both the HIV-1 genome and other target DNAs, both in HIV and other retroviruses.

In certain embodiments, the Cpf1 nucleases and gRNAs are encoded in isolated nucleic acid sequences, which are delivered to cells including the target gene for expression in situ. The isolated nucleic acid sequences can be included in any suitable expression vector, for expression in a particular cell type. Alternatively, they can be expressed as polypeptides in any suitable in vivo or in vitro translation system, and delivered to host cells in microdelivery vehicles such as liposomes and the like. The polypeptides can be generated by a variety of methods including, for example, recombinant techniques or chemical synthesis. Once generated, polypeptides can be isolated and purified to any desired extent by means well known in the art. For example, one can use lyophilization following, for example, reversed phase (preferably) or normal phase HPLC, or size exclusion or partition chromatography on polysaccharide gel media such as Sephadex G-25. The composition of the final polypeptide may be confirmed by amino acid analysis after degradation of the peptide by standard means, by amino acid sequencing, or by FAB-MS techniques. Salts, including acid salts, esters, amides, and N-acyl derivatives of an amino group of a polypeptide may be prepared using methods known in the art, and such peptides are useful in the context of the present invention.

The Cpf1 endonucleases of the present invention can have a nucleotide sequence identical that of wild type Acidaminococcus sp. BV3L6 or of Lachnospiraceae bacterium ND2006 (Zetsche B. et al., Cell 163, 1-13 Oct. 22, 2015). Alternatively, the Cpf1 of any species can be utilized, if it can be shown to mediate gRNA guided gene editing in a particular cell type or individual animal.

The wild type Acidaminococcus or Lachnospiraceae Cpf1 sequences can be modified to encode biologically active variants of Cpf1, and these variants can have or can include, for example, an amino acid sequence that differs from a wild type Cpf1 by virtue of containing one or more mutations (e.g., an addition, deletion, or substitution mutation or a combination of such mutations). The Cpf1 nucleotide sequence can be modified to encode biologically active variants of Cpf1, and these variants can have or can include, for example, an amino acid sequence that differs from a wild type Cpf1 by virtue of containing one or more mutations (e.g., an addition, deletion, or substitution mutation or a combination of such mutations). One or more of the substitution mutations can be a substitution (e.g., a conservative amino acid substitution). For example, a biologically active variant of a Cpf1 polypeptide can have an amino acid sequence with at least or about 50% sequence identity (e.g., at least or about 50%, 55%, 60%, 65%, 70%, 75%, 80%, 85%, 90%, 95%, 97%, 98%, or 99% sequence identity) to a wild type Cpf1 polypeptide. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. The amino acid residues in the Cpf1 amino acid sequence can be non-naturally occurring amino acid residues. Naturally occurring amino acid residues include those naturally encoded by the genetic code as well as non-standard amino acids (e.g., amino acids having the D-configuration instead of the L-configuration). The present peptides can also include amino acid residues that are modified versions of standard residues (e.g. pyrrolysine can be used in place of lysine and selenocysteine can be used in place of cysteine). Non-naturally occurring amino acid residues are those that have not been found in nature, but that conform to the basic formula of an amino acid and can be incorporated into a peptide. These include D-alloisoleucine(2R,3S)-2-amino-3-methylpentanoic acid and L-cyclopentyl glycine (S)-2-amino-2-cyclopentyl acetic acid. For other examples, one can consult textbooks or the worldwide web (a site is currently maintained by the California Institute of Technology and displays structures of non-natural amino acids that have been successfully incorporated into functional proteins).

For example, the nucleic acid sequence of Cpf1 can be codon optimized for efficient expression in mammalian cells, i.e., “humanized” (Zetsche, et al., 2015). The Cpf1 endonuclease can be modified to serve as a “nickase”.

The Cpf1 nuclease sequence can be mutated to behave as “nickase”, which nicks rather than cleaves DNA, to yield single-stranded breaks. In Cas9, nickase activity is accomplished by mutations in the conserved HNH and RuvC domains, which are involved in strand specific cleavage. For example, an aspartate-to-alanine (D10A) mutation in the RuvC catalytic domain allows the Cas9 nickase mutant (Cas9n) to nick rather than cleave DNA to yield single-stranded breaks (Sander J. D. and Joung L. K., Nature Biotech 32, 347-355 (2014)). The Cpf1's of Acidaminococcus and Lachnospiraceae lack an HNH domain but do include a RuvC domain, so it is likely that a nickase Cpf1 can be created by mutations similar to those employed in Cas9. The biological activity of mutant Cpf1 can be assessed in ways known to one of ordinary skill in the art and includes, without limitation, in vitro cleavage assays or functional assays.

The Cpf1 nuclease sequence can also be mutated to produce a catalytically-deficient Cpf1. A catalytically deficient Cpf1 can be created by suitable mutation of the RuvC domain, as has been accomplished for Cas9 (Gilbert L. A. et al. Cell 154,442-51 (2013)). A catalytically defective Cpf1 is useful to localize fluorescent labels or regulatory proteins to specific target sites on a DNA molecule.

The Cpf1 nuclease sequence can be mutated to produce a Cpf1 with improved targeting efficiency and/or prevents off-targeting of the molecule as compared to the wild-type Cpf1. The Cpf1 molecule can comprise one or more mutations in the Cpf1 nuclease sequence which include, without limitation deletions, substitutions, modified nucleobases, locked nucleic acids, peptide nucleic acids, and the like.

The present invention also includes all homologs and orthologues of Cpf1, across all classes of the phyla bacteria and archaea, for example species included in the phylogeny shown in FIG. 2 of Haft D. H., et al. PLoS Comput Biol 1, 0474-0483 (2005). These homologs and orthologues are also included as variant and mutant forms, as previously stated. Cpf1 orthologues, include for example, Cpf1 from Acidaminococcus sp. BV3L6 and Lachnospiraceae bacterium ND 2006 (AsCpf1 and LbCpf1 respectively. These orthologues generally recognize TTTN PAMs that are positioned 5′ to the protospacer.

Guide Nucleic Acid Sequences:

Guide RNA sequences according to the present invention can be sense or anti-sense sequences. The specific sequence of the gRNA may vary, but, regardless of the sequence, useful guide RNA sequences will be those that minimize off-target effects while achieving high efficiency and complete ablation of the genomically integrated HIV-1 provirus. The length of the guide RNA sequence can vary from about 20 to about 60 or more nucleotides, for example about 20, about 21, about 22, about 23, about 24, about 25, about 26, about 27, about 28, about 29, about 30, about 31, about 32, about 33, about 34, about 35, about 36, about 37, about 38, about 39, about 40, about 45, about 50, about 55, about 60 or more nucleotides.

The guide RNA sequence can be configured as a single sequence or as a combination of one or more different sequences, e.g., a multiplex configuration. Multiplex configurations can include combinations of two, three, four, five, six, seven, eight, nine, ten, or more different guide RNAs.

The compositions and methods of the present invention may include a sequence encoding a guide RNA that is complementary to a target sequence in HIV. The genetic variability of HIV is reflected in the multiple groups and subtypes that have been described. A collection of HIV sequences is compiled in the Los Alamos HIV databases and compendiums (i.e., the sequence database web site is http://www.hiv.lani.gov). The methods and compositions of the invention can be applied to HIV from any of those various groups, subtypes, and circulating recombinant forms. These include for example, the HIV-1 major group (often referred to as Group M) and the minor groups, Groups N, O, and P, as well as but not limited to, any of the following subtypes, A, B, C, D, F, G, H, J and K. or group (for example, but not limited to any of the following Groups, N, O and P) of HIV.

The guide RNA can be a sequence complimentary to a coding or a non-coding sequence (i.e., a target sequence). For example, the guide RNA can be a sequence that is complementary to a HIV long terminal repeat (LTR) region. The gRNA sequences according to the present invention can be complementary to either the sense or anti-sense strands of the target sequences. They can include additional 5′ and/or 3′ sequences that may or may not be complementary to a target sequence. They can have less than 100% complementarity to a target sequence, for example 75% complementarity. When the compositions of the present invention are administered as an isolated nucleic acid or are contained within an expression vector, the Cpf1 endonuclease can be encoded by the same nucleic acid or vector as the gRNA sequences. Alternatively, or in addition, the Cpf1 endonuclease can be encoded in a physically separate nucleic acid from the gRNA sequences or in a separate vector.

Isolated Nucleic Acid Sequences.

Isolated nucleic acid molecules can be produced by standard techniques. For example, polymerase chain reaction (PCR) techniques can be used to obtain an isolated nucleic acid containing a nucleotide sequence described herein, including nucleotide sequences encoding a polypeptide described herein. PCR can be used to amplify specific sequences from DNA as well as RNA, including sequences from total genomic DNA or total cellular RNA. Various PCR methods are described in, for example, PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >50-100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids of the invention also can be obtained by mutagenesis of, e.g., a naturally occurring portion of a Cas9-encoding DNA (in accordance with, for example, the formula above).

It will be understood that all CRISPR/Cpf1 compositions or methods of the present invention can be combined with those of a CRISPR/Cas9 system, to obtain the target sequence spectrum of both systems.

Modified or Mutated Nucleic Acid Sequences:

In some embodiments, any of the nucleic acid sequences embodied herein (e.g. mutated Cpf1 to improve target efficiency and/or prevent off-target effects) may be modified or derived from a native nucleic acid sequence, for example, by introduction of mutations, deletions, substitutions, modification of nucleobases, backbones and the like. The nucleic acid sequences include the vectors, gene-editing agents, isolated nucleic acids, gRNAs, tracrRNA etc. The nucleic acid sequences of the present invention also include variants in which a different base is present at one or more of the nucleotide positions in the compound. For example, if the first nucleotide is an adenosine, variants may be produced which contain thymidine, guanosine or cytidine at this position. This may be done at any of the positions of the isolated nucleic acid sequence. The nucleic acid sequences of the invention may have modifications to the nucleobases or backbones. Examples of some modified nucleic acid sequences envisioned for this invention include those comprising modified backbones, for example, phosphorothioates, phosphotriesters, methyl phosphonates, short chain alkyl or cycloalkyl intersugar linkages or short chain heteroatomic or heterocyclic intersugar linkages. In some embodiments, modified oligonucleotides comprise those with phosphorothioate backbones and those with heteroatom backbones, CH₂—NH—O—CH₂, CH, —N(CH₃)—O—CH₂ [known as a methylene(methylimino) or MMI backbone], CH₂—O—N(CH₃)—CH₂, CH₂—N(CH₃)—N(CH₃)—CH₂ and O—N(CH₃)—CH₂—CH₂ backbones, wherein the native phosphodiester backbone is represented as O—P—O—CH). The amide backbones disclosed by De Mesmaeker et al. Acc. Chem. Res. 1995, 28:366-374) are also embodied herein. In some embodiments, the nucleic acid sequences having morpholino backbone structures (Summerton and Weller, U.S. Pat. No. 5,034,506), peptide nucleic acid (PNA) backbone wherein the phosphodiester backbone of the oligonucleotide is replaced with a polyamide backbone, the nucleobases being bound directly or indirectly to the aza nitrogen atoms of the polyamide backbone (Nielsen et al. Science 1991, 254, 1497). The nucleic acid sequences may also comprise one or more substituted sugar moieties. The nucleic acid sequences may also have sugar mimetics such as cyclobutyls in place of the pentofuranosyl group.

The nucleic acid sequences may also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N₆ (6-aminohexyl)adenine and 2,6-diaminopurine. Kornberg, A., DNA Replication, W. H. Freeman & Co., San Francisco, 1980, pp 75-77; Gebeyehu, G., et al. Nucl. Acids Res. 1987, 15:4513). A “universal” base known in the art, e.g., inosine may be included. 5-Me-C substitutions have been shown to increase nucleic acid duplex stability by 0.6-1.2° C. (Sanghvi, Y. S., in Crooke, S. T. and Lebleu, B., eds., Antisense Research and Applications, CRC Press, Boca Raton, 1993, pp. 276-278).

Another modification of the nucleic acid sequences of the invention involves chemically linking to the nucleic acid sequences one or more moieties or conjugates which enhance the activity or cellular uptake of the oligonucleotide. Such moieties include but are not limited to lipid moieties such as a cholesterol moiety, a cholesteryl moiety (Letsinger et al., Proc. Natl. Acad. Sci. USA 1989, 86, 6553), cholic acid (Manoharan et al. Bioorg. Med. Chem. Let. 1994, 4, 1053), a thioether, e.g., hexyl-S-tritylthiol (Manoharan et al. Ann. N.Y. Acad. Sci. 1992, 660, 306; Manoharan et al. Bioorg. Med. Chem. Let. 1993, 3, 2765), a thiocholesterol (Oberhauser et al., Nucl. Acids Res. 1992, 20, 533), an aliphatic chain, e.g., dodecandiol or undecyl residues (Saison-Behmoaras et al. EMBO J. 1991, 10, 111; Kabanov et al. FEBS Lett. 1990, 259, 327; Svinarchuk et al. Biochimie 1993, 75, 49), a phospholipid, e.g., di-hexadecyl-rac-glycerol or triethylammonium 1,2-di-O-hexadecyl-rac-glycero-3-H-phosphonate (Manoharan et al. Tetrahedron Lett. 1995, 36, 3651; Shea et al. Nucl. Acids Res. 1990, 18, 3777), a polyamine or a polyethylene glycol chain (Manoharan et al. Nucleosides & Nucleotides 1995, 14, 969), or adamantane acetic acid (Manoharan et al. Tetrahedron Lett. 1995, 36, 3651).

In another preferred embodiment, an isolated nucleic acid sequence, e.g. Cpf1 comprises combinations of phosphorothioate internucleotide linkages and at least one internucleotide linkage selected from the group consisting of: alkylphosphonate, phosphorodithioate, alkylphosphonothioate, phosphoramidate, carbamate, carbonate, phosphate triester, acetamidate, carboxymethyl ester, and/or combinations thereof. In another preferred embodiment, an isolated nucleic acid sequence optionally comprises at least one modified nucleobase comprising, peptide nucleic acids, locked nucleic acid (LNA) molecules, analogues, derivatives and/or combinations thereof.

It is not necessary for all positions in a given nucleic acid sequence to be uniformly modified, and in fact more than one of the aforementioned modifications may be incorporated in a single nucleic acid sequence or even at within a single nucleoside within a nucleic acid sequence.

Certain preferred isolated nucleic acid sequences of this invention are chimeric molecules. “Chimeric molecules” or “chimeras,” in the context of this invention, are isolated nucleic acid sequences which contain two or more chemically distinct regions, each made up of at least one nucleotide. These isolated nucleic acid sequences typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of antisense modulation of gene expression. Consequently, comparable results can often be obtained with shorter isolated nucleic acid sequences when chimeric isolated nucleic acid sequences are used, compared to phosphorothioate deoxyoligonucleotides hybridizing to the same target region.

Chimeric isolated nucleic acid sequences of the invention may be formed as composite structures of two or more oligonucleotides, modified oligonucleotides, oligonucleosides and/or oligonucleotide mimetics as described above. Such; compounds have also been referred to in the art as hybrids or gapmers. Representative United States patents that teach the preparation of such hybrid structures comprise, but are not limited to, U.S. Pat. Nos. 5,013,830; 5,149,797; 5, 220,007; 5,256,775; 5,366,878; 5,403,711; 5,491,133; 5,565,350; 5,623,065; 5,652,355; 5,652,356; and 5,700,922, each of which is herein incorporated by reference.

In another embodiment, the region of the isolated nucleic acid sequence which is modified comprises at least one nucleotide modified at the 2′ position of the sugar, most preferably a 2′-O-alkyl, 2′-O-alkyl-O-alkyl or 2′-fluoro-modified nucleotide. In another embodiment, the isolated nucleic acid sequences can also be modified to enhance nuclease resistance. Cells contain a variety of exo- and endonucleases which can degrade nucleic acids. A number of nucleotide and nucleoside modifications have been shown to make nucleic acid sequence into which they are incorporated more resistant to nuclease digestion than the native oligodeoxynucleotide. Nuclease resistance is routinely measured by incubating isolated nucleic acid sequences with cellular extracts or isolated nuclease solutions and measuring the extent of intact oligonucleotide remaining over time, usually by gel electrophoresis. Isolated nucleic acid sequences which have been modified to enhance their nuclease resistance survive intact for a longer time than unmodified isolated nucleic acid sequences. A variety of oligonucleotide modifications have been demonstrated to enhance or confer nuclease resistance. Isolated nucleic acid sequences can contain at least one phosphorothioate modification. In some cases, oligonucleotide modifications which enhance target binding affinity are also, independently, able to enhance nuclease resistance. Some desirable modifications can be found in De Mesmaeker et al. Acc. Chem. Res. 1995, 28:366-374.

In some embodiments, the RNA molecules e.g. crRNA, tracrRNA, gRNA, are engineered to comprise one or more modified nucleobases. For example, known modifications of RNA molecules can be found, for example, in Genes VI, Chapter 9 (“Interpreting the Genetic Code”), Lewis, ed. (1997, Oxford University Press, New York), and Modification and Editing of RNA, Grosjean and Benne, eds. (1998, ASM Press, Washington D.C.). Modified RNA components include the following: 2′-O-methylcytidine; N⁴-methylcytidine; N⁴-2′-O-dimethylcytidine; N⁴-acetylcytidine; 5-methylcytidine; 5,2′-O-dimethylcytidine; 5-hydroxymethylcytidine; 5-formylcytidine; 2′-O-methyl-5-formaylcytidine; 3-methylcytidine; 2-thiocytidine; lysidine; 2′-O-methyluridine; 2-thiouridine; 2-thio-2′-O-methyluridine; 3,2′-O-dimethyluridine; 3-(3-amino-3-carboxypropyl)uridine; 4-thiouridine; ribosylthymine; 5,2′-O-dimethyluridine; 5-methyl-2-thiouridine; 5-hydroxyuridine; 5-methoxyuridine; uridine 5-oxyacetic acid; uridine 5-oxyacetic acid methyl ester; 5-carboxymethyluridine; 5-methoxycarbonylmethyluridine; 5-methoxycarbonylmethyl-2′-O-methyluridine; 5-methoxycarbonylmethy 1-2′-thiouridine; 5-carbamoylmethyluridine; 5-carbamoylmethyl-2′-O-methyluridine; 5-(carboxyhydroxymethyl)uridine; 5-(carboxyhydroxymethyl) uridinemethyl ester; 5-aminomethy 1-2-thiouridine; 5-methylaminomethyluridine; 5-methylaminomethyl-2-thiouridine; 5-methylaminomethy 1-2-selenouridine; 5-carboxymethylaminomethyluridine; 5-carboxymethylaminomethyl-2′-O-methyluridine; 5-carboxymethylaminomethy 1-2-thiouridine; dihydrouridine; dihydroribosylthymine; 2′-methyladenosine; 2-methyladenosine; N⁶Nmethyladenosine; N⁶, N⁶-dimethyladenosine; N⁶,2′-O-trimethyladenosine; 2 methylthio-N⁶Nisopentenyladenosine; N⁶-(cis-hydroxyisopentenyl)-adenosine; 2-methylthio-N⁶-(cis-hydroxyisopentenyl)-adenosine; N⁶-glycinylcarbamoyl)adenosine; N⁶ threonylcarbamoyl adenosine; N⁶-methyl-N⁶-threonylcarbamoyl adenosine; 2-methylthio-N⁶-methyl-N⁶-threonylcarbamoyl adenosine; N⁶-hydroxynorvalylcarbamoyl adenosine; 2-methylthio-N⁶-hydroxnorvalylcarbamoyl adenosine; 2′-O-ribosyladenosine (phosphate); inosine; 2′O-methyl inosine; 1-methyl inosine; 1;2′-O-dimethyl inosine; 2′-O-methyl guanosine; 1-methyl guanosine; N²-methyl guanosine; N², N²-dimethyl guanosine; N², 2′-O-dimethyl guanosine; N², N², 2′-O-trimethyl guanosine; 2′-O-ribosyl guanosine (phosphate); 7-methyl guanosine; N²;7-dimethyl guanosine; N²; N²;7-trimethyl guanosine; wyosine; methylwyosine; under-modified hydroxywybutosine; wybutosine; hydroxywybutosine; peroxywybutosine; queuosine; epoxyqueuosine; galactosyl-queuosine; mannosyl-queuosine; 7-cyano-7-deazaguanosine; arachaeosine [also called 7-formamido-7-deazaguanosine]; and 7-aminomethyl-7-deazaguanosine.

In other embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tₘ(i.e., higher target binding affinity) than 2′-deoxyoligonucleotides against a given target.

Methods for Preventing and Treating a Viral Infection.

A primary HIV-1 infection subsides within a few weeks to a few months, and is typically followed by a long clinical “latent” period which may last for up to 10 years. The subject's CD4 lymphocyte numbers rebound, but not to pre-infection levels and most subjects undergo seroconversion, that is, they have detectable levels of anti-HIV-1 antibody in their blood, within 2 to 4 weeks of infection. During the latent period, also referred to as the clinical latency stage, people who are infected with HIV may experience no HIV-related symptoms, or only mild ones. But, the HIV-1 virus continues to reproduce at very low levels. In subjects who have treated with anti-retroviral therapies, this latent period may extend for several decades or more. However, subjects at this stage are still able to transmit HIV to others even if they are receiving antiretroviral therapy, although anti-retroviral therapy reduces the risk of transmission. Anti-retroviral therapy does not suppress low levels of viral genome expression nor does it efficiently target latently infected cells such as resting memory T cells, brain macrophages, microglia, astrocytes and gut associated lymphoid cells.

Latent infection by integrated virus is a characteristic of retroviruses, and is also seen in many other types of virus, including polyoma virus, herpes virus, hepatitis virus B, and human papilloma virus. There is a need for treatments that will inactivate or excise integrated proviral DNA from host cell genomes, or prevent integration of proviral DNA in the first place.

Therefore, the present invention includes a composition for use in inactivating an integrated proviral DNA in the genome of a host cell in vitro or in vivo. The composition includes at least one isolated nucleic acid sequence encoding a Cpf1 endonuclease, and at least one gRNA. The at least one gRNA is complementary to a target DNA sequence in the proviral DNA.

The present invention also includes a method of inactivating an integrated proviral DNA in the genome of a host cell in vitro or in vivo, including the steps of: treating the host cell with at least one isolated nucleic acid sequence encoding a Cpf1 endonuclease; treating the host cell with at least one isolated nucleic acid sequence encoding a gRNA, the at least one gRNA being complementary to a target sequence in the proviral DNA; and inactivating the proviral DNA.

The present invention also provides a method of preventing a viral infection of host cells of a patient at risk of retroviral infection. The method includes the steps of determining that a patient is at risk of a viral infection of host cells; exposing the patient's host cells to an effective amount of an expression vector composition including an isolated nucleic acid encoding a Cpf1 endonuclease, and at least one gRNA that is complementary to a target sequence in the viral genome; stably expressing the Cpf1 endonuclease and the at least one gRNA in the host cells; and preventing viral infection of the host cells.

In the case of integrated HIV, e.g. HIV-1, useful gRNAs have been developed, which are complementary to the U3, R, or U5 region of the HIV-1 LTR (Hu W. et al., Proc Natl Acad Sci USA 111, 11461-11466 (2014)). The gRNAs are effective at eradicating integrated proviral HIV-1. Stable expression of the gRNAs, together with stable expression of Cas9 prevents new infection of T cells with HIV-1 (Hu W. et al., Proc Natl Acad Sci USA 111, 11461-11466 (2014)). The gRNAs were developed for use with Cas9, but it is likely that gRNAs complementary to target sequences adjacent to Cpf1 PAMs is also be effective in eradicating or preventing latent HIV-1 infection.

Exemplary target sequences in the HIV-1 LTR, adjacent to PAMs for Cpf1, are disclosed in Example 1. gRNAs can similarly be identified by their adjacency to Cpf1 PAMs in other viruses, including, but not limited to, human immunodeficiency virus-1 (HIV-1), human immunodeficiency virus-2 (HIV-2), human T cell lymphotropic virus type I (HTLV-1), human T cell lymphotropic virus type II (HTLV-II), herpes simplex virus type 1 (HSV-1), herpes simplex virus type 2 (HSV-2), and JC virus (JCV). Inactivation or excision of JCV from host oligodendrocytes will be of great use in the therapy of progressive multifocal leukoencephalopathy (PML).

Recombinant Constructs and Delivery Vehicles.

Exemplary expression vectors for inclusion in the pharmaceutical composition include plasmid vectors and lentiviral vectors, but the present invention is not limited to these vectors. A wide variety of host/expression vector combinations may be used to express the nucleic acid sequences described herein. Suitable expression vectors include, without limitation, plasmids and viral vectors derived from, for example, bacteriophage, baculoviruses, and retroviruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.). A marker gene can confer a selectable phenotype on a host cell. For example, a marker can confer biocide resistance, such as resistance to an antibiotic (e.g., kanamycin, G418, bleomycin, or hygromycin). An expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or FLAG™ tag (Kodak, New Haven, Conn.) sequences typically are expressed as a fusion with the encoded polypeptide. Such tags can be inserted anywhere within the polypeptide, including at either the carboxyl or amino terminus. The vector can also include origins of replication, scaffold attachment regions (SARs), regulatory regions and the like. The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, nuclear localization signals, and introns. The term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically comprises at least a core (basal) promoter. A promoter also may include at least one control element, such as an enhancer sequence, an upstream element or an upstream activation region (UAR). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence. Suitable promoters which may be employed include, but are not limited to, the retroviral LTR; the SV40 promoter; and the human cytomegalovirus (CMV) promoter described in Miller, et al., Biotechniques, Vol. 7, No. 9, 980-990 (1989), or any other promoter (e.g., cellular promoters such as eukaryotic cellular promoters including, but not limited to, the histone, pol III, and β-actin promoters). Other viral promoters which may be employed include, but are not limited to, adenovirus promoters, TK promoters, and B19 parvovirus promoters.

Expression of the Cpf1/guide nucleic acid sequences may be controlled by any promoter/enhancer element known in the art, but these regulatory elements must be functional in the host selected for expression. Promoters which may be used to control gene expression include, but are not limited to, cytomegalovirus (CMV) promoter (U.S. Pat. Nos. 5,385,839 and 5,168,062), the SV40 early promoter region (Benoist and Chambon, 1981, Nature 290:304-310), the promoter contained in the 3′ long terminal repeat of Rous sarcoma virus (Yamamoto, et al., Cell 22:787-797, 1980), the herpes thymidine kinase promoter (Wagner et al., Proc. Natl. Acad. Sci. U.S.A. 78:1441-1445, 1981), the regulatory sequences of the metallothionein gene (Brinster et al., Nature 296:39-42, 1982); prokaryotic expression vectors such as the β-lactamase promoter (Villa-Kamaroff, et al., Proc. Natl. Acad. Sci. U.S.A. 75:3727-3731, 1978), or the tac promoter (DeBoer, et al., Proc. Natl. Acad. Sci. U.S.A. 80:21-25, 1983); see also “Useful proteins from recombinant bacteria” in Scientific American, 242:74-94, 1980; promoter elements from yeast or other fungi such as the Gal 4 promoter, the ADC (alcohol dehydrogenase) promoter, PGK (phosphoglycerol kinase) promoter, alkaline phosphatase promoter; and the animal transcriptional control regions, which exhibit tissue specificity and have been utilized in transgenic animals: elastase I gene control region which is active in pancreatic acinar cells (Swift et al., Cell 38:639-646, 1984; Ornitz et al., Cold Spring Harbor Symp. Quant. Biol. 50:399-409, 1986; MacDonald, Hepatology 7:425-515, 1987); insulin gene control region which is active in pancreatic beta cells (Hanahan, Nature 315:115-122, 1985), immunoglobulin gene control region which is active in lymphoid cells (Grosschedl et al., Cell 38:647-658, 1984; Adames et al., Nature 318:533-538, 1985; Alexander et al., Mol. Cell. Biol. 7:1436-1444, 1987), mouse mammary tumor virus control region which is active in testicular, breast, lymphoid and mast cells (Leder et al., Cell 45:485-495, 1986), albumin gene control region which is active in liver (Pinkert et al., Genes and Devel. 1:268-276, 1987), alpha-fetoprotein gene control region which is active in liver (Krumlauf et al., Mol. Cell. Biol. 5:1639-1648, 1985; Hammer et al., Science 235:53-58, 1987), alpha 1-antitrypsin gene control region which is active in the liver (Kelsey et al., Genes and Devel. 1: 161-171, 1987), beta-globin gene control region which is active in myeloid cells (Mogram et al., Nature 315:338-340, 1985; Kollias et al., Cell 46:89-94, 1986), myelin basic protein gene control region which is active in oligodendrocyte cells in the brain (Readhead et al., Cell 48:703-712, 1987), myosin light chain-2 gene control region which is active in skeletal muscle (Sani, Nature 314:283-286, 1985), and gonadotropic releasing hormone gene control region which is active in the hypothalamus (Mason et al., Science 234:1372-1378, 1986).

In another embodiment the invention comprises an inducible promoter. One such promoter is the tetracycline-controlled transactivator (tTA)-responsive promoter (tet system), a prokaryotic inducible promoter system which has been adapted for use in mammalian cells. The tet system was organized within a retroviral vector so that high levels of constitutively-produced tTA mRNA function not only for production of tTA protein but also the decreased basal expression of the response unit by antisense inhibition. See, Paulus, W. et al., “Self-Contained, Tetracycline-Regulated Retroviral Vector System for Gene Delivery to Mammalian Cells”, J of Virology, January. 1996, Vol. 70, No. 1, pp. 62-67. The selection of a suitable promoter will be apparent to those skilled in the art from the teachings contained herein.

The present invention provides expression vectors for use in inactivating target genes the genome of a host cell. Each expression vector includes at least one isolated nucleic acid sequence encoding a Cpf1 endonuclease, and at least one (gRNA), with the at least one gRNA being complementary to a target sequence in the target gene. A nucleic acid sequence encoding the least one Cpf1 endonuclease, and a nucleic acid sequence encoding at least one gRNA, can be included in a single expression vector, or in separate vectors.

A preferred vector for expressing Cpf1 systems in mammalian cells is a lentiviral vector, because of its high transduction efficiency and low toxicity. Other suitable expression vectors include, without limitation, plasmids and viral vectors derived from, for example, bacteriophage, baculoviruses, retroviruses. adenoviruses (“Ad”), adeno-associated viruses (AAV), and vesicular stomatitis virus (VSV), and pox viral vectors such as avipox or orthopox vectors. Additional expression vectors also can include derivatives of SV40 and known bacterial plasmids, e.g., E. coli plasmids col E1, pCR1, pBR322, pMal-C2, pET, pGEX, pMB9 and their derivatives; plasmids such as RP4; phage DNAs, e.g., the numerous derivatives of phage 1, e.g., NM989, and other phage DNA, e.g., M13 and filamentous single stranded phage DNA; yeast plasmids such as the 2μ plasmid or derivatives thereof; and vectors derived from combinations of plasmids and phage DNAs, such as plasmids that have been modified to employ phage DNA or other expression control sequences.

Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.). Suitable promoters and enhancers can be included in the vectors, with the selection being made according to the cell type in which expression is desired, by experimental means well known in the art.

The polynucleotides of the invention may also be used with a microdelivery vehicle such as cationic liposomes and adenoviral vectors. For a review of the procedures for liposome preparation, targeting and delivery of contents, see Mannino and Gould-Fogerite, BioTechniques, 6:682 (1988). See also, Feigner and Holm, Bethesda Res. Lab. Focus, 11(2):21 (1989) and Maurer, R. A., Bethesda Res. Lab. Focus, 11(2):25 (1989).

Therefore, the present invention encompasses a lentiviral vector composition for inactivating proviral DNA integrated into the genome of a host cell latently infected with HIV. The composition includes an isolated nucleic acid encoding an endonuclease, and at least one isolated nucleic acid encoding at least one guide gRNA including a spacer sequence that is complementary to a target sequence in a proviral HIV DNA, with the isolated nucleic acids being included in at least one lentiviral expression vector. The lentiviral expression vector induces the expression of the endonuclease and the at least one gRNA in a host cell.

All of the isolated nucleic acids can be included in a single lentiviral expression vector, or the nucleic acids can be subdivided into any suitable combination of lentiviral vectors. For example, the endonuclease can be incorporated into a first lentiviral expression vector, a first gRNA can be incorporated into a second lentiviral expression vector, and a second gRNA can be incorporated into a third lentiviral expression vector. When multiple expression vectors are used, it is not necessary all of them be lentiviral vectors.

Recombinant constructs are also provided herein and can be used to transform cells. A recombinant nucleic acid construct comprises a nucleic acid encoding a Cpf1 and/or a guide RNA complementary to a target sequence in HIV as described herein, operably linked to a regulatory region suitable for expressing the Cpf1 and/or a guide RNA complementary to a target sequence in HIV in the cell. It will be appreciated that a number of nucleic acids can encode a polypeptide having a particular amino acid sequence. The degeneracy of the genetic code is well known in the art. For many amino acids, there is more than one nucleotide triplet that serves as the codon for the amino acid. For example, codons in the coding sequence for Cpf1 can be modified such that optimal expression in a particular organism is obtained, using appropriate codon bias tables for that organism.

Several delivery methods may be utilized in conjunction with the molecules embodied herein for in vitro (cell cultures) and in vivo (animals and patients) systems. In one embodiment, a lentiviral gene delivery system may be utilized. Such a system offers stable, long term presence of the gene in dividing and non-dividing cells with broad tropism and the capacity for large DNA inserts. (Dull et al, J Virol, 72:8463-8471 1998). In an embodiment, adeno-associated virus (AAV) may be utilized as a delivery method. AAV is a non-pathogenic, single-stranded DNA virus that has been actively employed in recent years for delivering therapeutic gene in in vitro and in vivo systems (Choi et al, Curr Gene Ther, 5:299-310, 2005).

In certain embodiments of the invention, non-viral vectors may be used to effectuate transfection. Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam and Lipofectin). Cationic and neutral lipids that are suitable for efficient receptor-recognition lipofection of polynucleotides include those described in U.S. Pat. No. 7,166,298 to Jessee or U.S. Pat. No. 6,890,554 to Jesse, the contents of each of which are incorporated by reference. Delivery can be to cells (e.g. in vitro or ex vivo administration) or target tissues (e.g. in vivo administration).

Synthetic vectors are typically based on cationic lipids or polymers which can complex with negatively charged nucleic acids to form particles with a diameter in the order of 100 nm. The complex protects nucleic acid from degradation by nuclease. Moreover, cellular and local delivery strategies have to deal with the need for internalization, release, and distribution in the proper subcellular compartment. Systemic delivery strategies encounter additional hurdles, for example, strong interaction of cationic delivery vehicles with blood components, uptake by the reticuloendothelial system, kidney filtration, toxicity and targeting ability of the carriers to the cells of interest. Modifying the surfaces of the cationic non-virals can minimize their interaction with blood components, reduce reticuloendothelial system uptake, decrease their toxicity and increase their binding affinity with the target cells. Binding of plasma proteins (also termed opsonization) is the primary mechanism for RES to recognize the circulating nanoparticles. For example, macrophages, such as the Kupffer cells in the liver, recognize the opsonized nanoparticles via the scavenger receptor.

The nucleic acid sequences of the invention can be delivered to an appropriate cell of a subject. This can be achieved by, for example, the use of a polymeric, biodegradable microparticle or microcapsule delivery vehicle, sized to optimize phagocytosis by phagocytic cells such as macrophages. For example, PLGA (poly-lacto-co-glycolide) microparticles approximately 1-10 μm in diameter can be used. The polynucleotide is encapsulated in these microparticles, which are taken up by macrophages and gradually biodegraded within the cell, thereby releasing the polynucleotide. Once released, the DNA is expressed within the cell. A second type of microparticle is intended not to be taken up directly by cells, but rather to serve primarily as a slow-release reservoir of nucleic acid that is taken up by cells only upon release from the micro-particle through biodegradation. These polymeric particles should therefore be large enough to preclude phagocytosis (i.e., larger than 5 μm and preferably larger than 20 μm). Another way to achieve uptake of the nucleic acid is using liposomes, prepared by standard methods. The nucleic acids can be incorporated alone into these delivery vehicles or co-incorporated with tissue-specific antibodies, for example antibodies that target cell types that are commonly latently infected reservoirs of HIV infections. Alternatively, one can prepare a molecular complex composed of a plasmid or other vector attached to poly-L-lysine by electrostatic or covalent forces. Poly-L-lysine binds to a ligand that can bind to a receptor on target cells. Delivery of “naked DNA” (i.e., without a delivery vehicle) to an intramuscular, intradermal, or subcutaneous site, is another means to achieve in vivo expression. In the relevant polynucleotides (e.g., expression vectors) the nucleic acid sequence encoding an isolated nucleic acid sequence comprising a sequence encoding Cpf1 and/or a guide RNA complementary to a target sequence of HIV, as described above.

In some embodiments, delivery of vectors can also be mediated by exosomes. Exosomes are lipid nanovesicles released by many cell types. They mediate intercellular communication by transporting nucleic acids and proteins between cells. Exosomes contain RNAs, miRNAs, and proteins derived from the endocytic pathway. They may be taken up by target cells by endocytosis, fusion, or both. Exosomes can be harnessed to deliver nucleic acids to specific target cells.

The expression constructs of the present invention can also be delivered by means of nanoclews. Nanoclews are a cocoon-like DNA nanocomposites (Sun, et al., J. Am. Chem. Soc. 2014, 136:14722-14725). They can be loaded with nucleic acids for uptake by target cells and release in target cell cytoplasm. Methods for constructing nanoclews, loading them, and designing release molecules can be found in Sun, et al. (Sun W, et al., J. Am. Chem. Soc. 2014, 136:14722-14725; Sun W, et al., Angew. Chem. Int. Ed. 2015: 12029-12033.)

The nucleic acids and vectors may also be applied to a surface of a device (e.g., a catheter) or contained within a pump, patch, or any other drug delivery device. The nucleic acids and vectors disclosed herein can be administered alone, or in a mixture, in the presence of a pharmaceutically acceptable excipient or carrier (e.g., physiological saline). The excipient or carrier is selected on the basis of the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

In some embodiments of the invention, liposomes are used to effectuate transfection into a cell or tissue. The pharmacology of a liposomal formulation of nucleic acid is largely determined by the extent to which the nucleic acid is encapsulated inside the liposome bilayer. Encapsulated nucleic acid is protected from nuclease degradation, while those merely associated with the surface of the liposome is not protected. Encapsulated nucleic acid shares the extended circulation lifetime and biodistribution of the intact liposome, while those that are surface associated adopt the pharmacology of naked nucleic acid once they disassociate from the liposome. Nucleic acids may be entrapped within liposomes with conventional passive loading technologies, such as ethanol drop method (as in SALP), reverse-phase evaporation method, and ethanol dilution method (as in SNALP).

Liposomal delivery systems provide stable formulation, provide improved pharmacokinetics, and a degree of ‘passive’ or ‘physiological’ targeting to tissues. Encapsulation of hydrophilic and hydrophobic materials, such as potential chemotherapy agents, are known. See for example U.S. Pat. No. 5,466,468 to Schneider, which discloses parenterally administrable liposome formulation comprising synthetic lipids; U.S. Pat. No. 5,580,571, to Hostetler et al. which discloses nucleoside analogues conjugated to phospholipids; U.S. Pat. No. 5,626,869 to Nyqvist, which discloses pharmaceutical compositions wherein the pharmaceutically active compound is heparin or a fragment thereof contained in a defined lipid system comprising at least one amphiphatic and polar lipid component and at least one nonpolar lipid component.

Liposomes and polymerosomes can contain a plurality of solutions and compounds. In certain embodiments, the complexes of the invention are coupled to or encapsulated in polymersomes. As a class of artificial vesicles, polymersomes are tiny hollow spheres that enclose a solution, made using amphiphilic synthetic block copolymers to form the vesicle membrane. Common polymersomes contain an aqueous solution in their core and are useful for encapsulating and protecting sensitive molecules, such as drugs, enzymes, other proteins and peptides, and DNA and RNA fragments. The polymersome membrane provides a physical barrier that isolates the encapsulated material from external materials, such as those found in biological systems. Polymerosomes can be generated from double emulsions by known techniques, see Lorenceau et al., 2005, Generation of Polymerosomes from Double-Emulsions, Langmuir 21(20):9183-6, incorporated by reference.

In some embodiments of the invention, non-viral vectors are modified to effectuate targeted delivery and transfection. PEGylation (i.e. modifying the surface with polyethyleneglycol) is the predominant method used to reduce the opsonization and aggregation of non-viral vectors and minimize the clearance by reticuloendothelial system, leading to a prolonged circulation lifetime after intravenous (i.v.) administration. PEGylated nanoparticles are therefore often referred as “stealth” nanoparticles. The nanoparticles that are not rapidly cleared from the circulation will have a chance to encounter infected cells.

In some embodiments of the invention, targeted controlled-release systems responding to the unique environments of tissues and external stimuli are utilized. Gold nanorods have strong absorption bands in the near-infrared region, and the absorbed light energy is then converted into heat by gold nanorods, the so-called “photothermal effect”. Because the near-infrared light can penetrate deeply into tissues, the surface of gold nanorod could be modified with nucleic acids for controlled release. When the modified gold nanorods are irradiated by near-infrared light, nucleic acids are released due to thermo-denaturation induced by the photothermal effect. The amount of nucleic acids released is dependent upon the power and exposure time of light irradiation.

Regardless of whether compositions are administered as nucleic acids or polypeptides, they are formulated in such a way as to promote uptake by the mammalian cell. Useful vector systems and formulations are described above. In some embodiments the vector can deliver the compositions to a specific cell type. The invention is not so limited however, and other methods of DNA delivery such as chemical transfection, using, for example calcium phosphate, DEAE dextran, liposomes, lipoplexes, surfactants, and perfluoro chemical liquids are also contemplated, as are physical delivery methods, such as electroporation, micro injection, ballistic particles, and “gene gun” systems.

In other embodiments, the compositions comprise a cell which has been transformed or transfected with one or more Cpf1 encoding vectors and gRNAs. In some embodiments, the methods of the invention can be applied ex vivo. That is, a subject's cells can be removed from the body and treated with the compositions in culture to excise, for example, HIV sequences and the treated cells returned to the subject's body. The cell can be the subject's cells or they can be haplotype matched or a cell line. The cells can be irradiated to prevent replication. In some embodiments, the cells are human leukocyte antigen (HLA)-matched, autologous, cell lines, or combinations thereof. In other embodiments the cells can be a stem cell. For example, an embryonic stem cell or an artificial pluripotent stem cell (induced pluripotent stem cell (iPS cell)). Embryonic stem cells (ES cells) and artificial pluripotent stem cells (induced pluripotent stem cell, iPS cells) have been established from many animal species, including humans. These types of pluripotent stem cells would be the most useful source of cells for regenerative medicine because these cells are capable of differentiation into almost all of the organs by appropriate induction of their differentiation, with retaining their ability of actively dividing while maintaining their pluripotency. iPS cells, in particular, can be established from self-derived somatic cells, and therefore are not likely to cause ethical and social issues, in comparison with ES cells which are produced by destruction of embryos. Further, iPS cells, which are self-derived cell, make it possible to avoid rejection reactions, which are the biggest obstacle to regenerative medicine or transplantation therapy.

Transduced cells are prepared for reinfusion according to established methods. After a period of about 2-4 weeks in culture, the cells may number between 1×10⁶ and 1×10¹⁰. In this regard, the growth characteristics of cells vary from patient to patient and from cell type to cell type. About 72 hours prior to reinfusion of the transduced cells, an aliquot is taken for analysis of phenotype, and percentage of cells expressing the therapeutic agent. For administration, cells of the present invention can be administered at a rate determined by the LD₅₀ of the cell type, and the side effects of the cell type at various concentrations, as applied to the mass and overall health of the patient. Administration can be accomplished via single or divided doses. Adult stem cells may also be mobilized using exogenously administered factors that stimulate their production and egress from tissues or spaces that may include, but are not restricted to, bone marrow or adipose tissues.

Therefore, the present invention encompasses a method of eliminating a proviral DNA integrated into the genome of ex vivo cultured host cells latently infected with HIV, wherein a proviral HIV DNA is integrated into the host cell genome. The method includes the steps of obtaining a population of host cells latently infected with HIV; culturing the host cells ex vivo; treating the host cells with a composition including a Cpf endonuclease, and at least one gRNA complementary to a target sequence in an LTR of the proviral HIV DNA; and eliminating the proviral DNA from the host cell genome. The same method steps are also useful for treating the donor of the latently infected host cell population when the following additional steps are added: producing an HIV-eliminated T cell population; infusing the HIV-eliminated T cell population into the patient; and treating the patient.

The compositions and methods that have proven effective for ex vivo treatment of latently infected T cells are very likely to be effective in vivo, if delivered by means of one or more suitable expression vectors. Therefore, the present invention encompasses a pharmaceutical composition for the inactivation of integrated HIV DNA in the cells of a mammalian subject, including an isolated nucleic acid sequence encoding an endonuclease, and at least one isolated nucleic acid sequence encoding at least one gRNA that is complementary to a target sequence in a proviral HIV DNA. Preferably, a combination of gRNA molecules is included. It is also preferable that the pharmaceutical composition also include at least one expression vector in which the isolated nucleic acid sequences are encoded.

Pharmaceutical Compositions.

In view of the previously stated utility of the CRISPR/Cpf1 in inactivating latent viruses, the present invention also provides a pharmaceutical composition for the inactivation of an integrated provirus in the cells of a mammalian subject. The composition includes an isolated nucleic acid sequence encoding a Cpf1 endonuclease; and at least one isolated nucleic acid sequence encoding at least one guide RNA (gRNA) that is complementary to a target sequence in a proviral provirus DNA. Preferably, the isolated nucleic acid sequences are included in at least one expression vector.

The present invention also provides a method of treating a mammalian subject infected with a virus The method includes the steps of: determining that a mammalian subject is infected with a virus, administering, to the subject, an effective amount of the previously stated pharmaceutical composition, and treating the subject for the viral infection.

In other embodiments, a method of inhibiting replication of a retrovirus, e.g. HIV in a cell or a subject, comprises contacting the cell or administering to the subject, a pharmaceutical composition comprising a therapeutically effective amount of an isolated nucleic acid sequence encoding a Cpf1 endonuclease; at least one guide RNA (gRNA), the gRNA being complementary to a target nucleic acid sequence in a retroviral genome, an anti-viral agent, or combinations thereof. In certain embodiments, a method of eradicating a retroviral genome in a cell or a subject, comprises contacting the cell or administering to the subject, a pharmaceutical composition comprising a therapeutically effective amount of a gene editing agent; at least one guide RNA (gRNA), the gRNA being complementary to a target nucleic acid sequence in a retroviral genome, an anti-viral agent, or combinations thereof. In addition, one or more therapeutic agents which alleviate any other symptoms that may be associated with the virus infection, e.g. fever, chills, headaches, secondary infections, can be administered in concert with, or as part of the pharmaceutical composition or at separate times. These agents comprise, without limitation, an anti-pyretic agent, anti-inflammatory agent, anti-fungal agent, anti-parasitic agent, chemotherapeutic agent, antibiotics, immunomodulating agent, or combinations thereof.

A therapeutically effective amount of a composition (i.e., an effective dosage) means an amount sufficient to produce a therapeutically (e.g., clinically) desirable result. The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions of the invention can include a single treatment or a series of treatments.

The pharmaceutical compositions of the present invention can be prepared in a variety of ways known to one of ordinary skill in the art. Regardless of their original source or the manner in which they are obtained, the compositions of the invention can be formulated in accordance with their use. For example, the nucleic acids and vectors described above can be formulated within compositions for application to cells in tissue culture or for administration to a patient or subject. Any of the pharmaceutical compositions of the invention can be formulated for use in the preparation of a medicament, and particular uses are indicated below in the context of treatment, e.g., the treatment of a subject having an HIV infection or at risk for contracting and HIV infection. When employed as pharmaceuticals, any of the nucleic acids and vectors can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal or intramuscular injection or infusion; or intracranial, e.g., intrathecal or intraventricular administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids, powders, and the like. Conventional pharmaceutical carriers, aqueous, powder or oily bases, thickeners and the like may be necessary or desirable.

This invention also includes pharmaceutical compositions which contain, as the active ingredient, nucleic acids and vectors described herein in combination with one or more pharmaceutically acceptable carriers. We use the terms “pharmaceutically acceptable” (or “pharmacologically acceptable”) to refer to molecular entities and compositions that do not produce an adverse, allergic or other untoward reaction when administered to an animal or a human, as appropriate. The term “pharmaceutically acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance. In making the compositions of the invention, the active ingredient is typically mixed with an excipient, diluted by an excipient or enclosed within such a carrier in the form of, for example, a capsule, tablet, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), lotions, creams, ointments, gels, soft and hard gelatin capsules, suppositories, sterile injectable solutions, and sterile packaged powders. As is known in the art, the type of diluent can vary depending upon the intended route of administration. The resulting compositions can include additional agents, such as preservatives. In some embodiments, the carrier can be, or can include, a lipid-based or polymer-based colloid. In some embodiments, the carrier material can be a colloid formulated as a liposome, a hydrogel, a microparticle, a nanoparticle, or a block copolymer micelle. As noted, the carrier material can form a capsule, and that material may be a polymer-based colloid.

The nucleic acid sequences of the invention can be delivered to an appropriate cell of a subject. This can be achieved by, for example, the use of a polymeric, biodegradable microparticle or microcapsule delivery vehicle, sized to optimize phagocytosis by phagocytic cells such as macrophages. For example, PLGA (poly-lacto-co-glycolide) microparticles approximately 1-10 μm in diameter can be used. The polynucleotide is encapsulated in these microparticles, which are taken up by macrophages and gradually biodegraded within the cell, thereby releasing the polynucleotide. Once released, the DNA is expressed within the cell. A second type of microparticle is intended not to be taken up directly by cells, but rather to serve primarily as a slow-release reservoir of nucleic acid that is taken up by cells only upon release from the micro-particle through biodegradation. These polymeric particles should therefore be large enough to preclude phagocytosis (i.e., larger than 5 μm and preferably larger than 20 μm). Another way to achieve uptake of the nucleic acid is using liposomes, prepared by standard methods. The nucleic acids can be incorporated alone into these delivery vehicles or co-incorporated with tissue-specific antibodies, for example antibodies that target cell types that are commonly latently infected reservoirs of HIV infection, for example, brain macrophages, microglia, astrocytes, and gut-associated lymphoid cells. Alternatively, one can prepare a molecular complex composed of a plasmid or other vector attached to poly-L-lysine by electrostatic or covalent forces. Poly-L-lysine binds to a ligand that can bind to a receptor on target cells. Delivery of “naked DNA” (i.e., without a delivery vehicle) to an intramuscular, intradermal, or subcutaneous site, is another means to achieve in vivo expression. In the relevant polynucleotides (e.g., expression vectors) the nucleic acid sequence encoding the isolated nucleic acid sequence comprising a sequence encoding a CRISPR-associated endonuclease and a guide RNA is operatively linked to a promoter or enhancer-promoter combination. Promoters and enhancers are described above.

In some embodiments, the compositions of the invention can be formulated as a nano particle, for example, nanoparticles comprised of a core of high molecular weight linear polyethylenimine (LPEI) complexed with DNA and surrounded by a shell of polyethyleneglycol-modified (PEGylated) low molecular weight LPEI.

The nucleic acids and vectors may also be applied to a surface of a device (e.g., a catheter) or contained within a pump, patch, or other drug delivery device. The nucleic acids and vectors of the invention can be administered alone, or in a mixture, in the presence of a pharmaceutically acceptable excipient or carrier (e.g., physiological saline). The excipient or carrier is selected on the basis of the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

In some embodiments, the compositions may be formulated as a topical gel for blocking sexual transmission of HIV. The topical gel can be applied directly to the skin or mucous membranes of the male or female genital region prior to sexual activity. Alternatively, or in addition the topical gel can be applied to the surface or contained within a male or female condom or diaphragm.

The present invention also encompasses a method of treating a mammalian subject infected with HIV, including the steps of: determining that a mammalian subject is infected with HIV, administering an effective amount of the previously stated pharmaceutical composition to the subject, and treating the subject for HIV infection.

Pharmaceutical compositions according to the present invention can be prepared in a variety of ways known to one of ordinary skill in the art. For example, the nucleic acids and vectors described above can be formulated in compositions for application to cells in tissue culture or for administration to a patient or subject. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal or intramuscular injection or infusion; or intracranial, e.g., intrathecal or intraventricular administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids, powders, and the like. Conventional pharmaceutical carriers, aqueous, powder or oily bases, thickeners and the like may be necessary or desirable.

This invention also includes pharmaceutical compositions which contain, as the active ingredient, nucleic acids and vectors described herein, in combination with one or more pharmaceutically acceptable carriers. The terms “pharmaceutically acceptable” (or “pharmacologically acceptable”) refer to molecular entities and compositions that do not produce an adverse, allergic or other untoward reaction when administered to an animal or a human, as appropriate. The term “pharmaceutically acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance. In making the compositions of the invention, the active ingredient is typically mixed with an excipient, diluted by an excipient or enclosed within such a carrier in the form of, for example, a capsule, tablet, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), lotions, creams, ointments, gels, soft and hard gelatin capsules, suppositories, sterile injectable solutions, and sterile packaged powders. As is known in the art, the type of diluent can vary depending upon the intended route of administration. The resulting compositions can include additional agents, such as preservatives. In some embodiments, the carrier can be, or can include, a lipid-based or polymer-based colloid. In some embodiments, the carrier material can be a colloid formulated as a liposome, a hydrogel, a microparticle, a nanoparticle, or a block copolymer micelle. As noted, the carrier material can form a capsule, and that material may be a polymer-based colloid.

In some embodiments, the compositions of the invention can be formulated as a nanoparticle, for example, nanoparticles comprised of a core of high molecular weight linear polyethylenimine (LPEI) complexed with DNA and surrounded by a shell of polyethyleneglycol modified (PEGylated) low molecular weight LPEI. In some embodiments, the compositions can be formulated as a nanoparticle encapsulating the compositions embodied herein. L-PEI has been used to efficiently deliver genes in vivo into a wide range of organs such as lung, brain, pancreas, retina, bladder as well as tumor. L-PEI is able to efficiently condense, stabilize and deliver nucleic acids in vitro and in vivo.

The nucleic acids and vectors may also be applied to a surface of a device (e.g., a catheter) or contained within a pump, patch, or any other drug delivery device. The nucleic acids and vectors of the invention can be administered alone, or in a mixture, in the presence of a pharmaceutically acceptable excipient or carrier (e.g., physiological saline). The excipient or carrier is selected on the basis of the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

In some embodiments, the compositions can be formulated as a nanoparticle encapsulating a nucleic acid encoding Cpf1 or a variant Cpf1 and at least one gRNA sequence complementary to a target HIV; or it can include a vector encoding these components. Alternatively, the compositions can be formulated as a nanoparticle encapsulating the endonuclease and/or the polypeptides encoded by one or more of the nucleic acid compositions of the present invention.

In methods of treatment of HIV infection, a subject can be identified using standard clinical tests, for example, immunoassays to detect the presence of HIV antibodies or the HIV polypeptide p24 in the subject's serum, or through HIV nucleic acid amplification assays. An amount of such a composition provided to the subject that results in a complete resolution of the symptoms of the infection, a decrease in the severity of the symptoms of the infection, or a slowing of the infection's progression is considered a therapeutically effective amount. The present methods may also include a monitoring step to help optimize dosing and scheduling as well as predict outcome. In some methods of the present invention, one can first determine whether a patient has a latent HIV infection, and then make a determination as to whether or not to treat the patient with one or more of the compositions described herein.

The compositions of the present invention, when stably expressed in potential host cells, reduce or prevent new infection by HIV. Accordingly, the present invention encompasses a method of preventing HIV infection of T cells of a patient at risk of HIV infection. The method includes the steps of determining that a patient is at risk of HIV infection; exposing T cells of the patient to an effective amount of an expression vector composition including an isolated nucleic acid encoding an endonuclease, and at least one isolated nucleic acid encoding at least one gRNA that is complementary to a target sequence in the HIV DNA; stably expressing in the T cells the endonuclease and the at least one gRNA; and preventing HIV infection of the T cells.

A subject at risk for having an HIV infection can be, for example, any sexually active individual engaging in unprotected sex, i.e., engaging in sexual activity without the use of a condom; a sexually active individual having another sexually transmitted infection; an intravenous drug user; or an uncircumcised man. A subject at risk for having an HIV infection can also be, for example, an individual whose occupation may bring him or her into contact with HIV-infected populations, e.g., healthcare workers or first responders. A subject at risk for having an HIV infection can be, for example, an inmate in a correctional setting or a sex worker, that is, an individual who uses sexual activity for income employment or nonmonetary items such as food, drugs, or shelter.

CRISPR/Cpf1 Compositions for Correcting Genetic Disease.

It is well known that CRISPR/Cas9 system can produce not only a break or excision at a DNA sequence, but also the subsequent splicing in of a desired DNA sequence (see, e.g., Doudna and Charpentier, Science 346, 1258096-1-1258096-9 (2014)). Single stranded oligonucleotides present in the vicinity of a Cas9 mediated cut can be inserted into the cut site by homology-directed repair. This process has proven successful in correcting genetic defects in a mouse model (Yin H. et al., Nature Biotech 32, 551-554 (2014)). If the CRISPR/Cas system, with its blunt ended cuts can be used to correct a genetic disease, then the CRISPR/Cpf1 system, which leaves sticky ends at a break, will prove even more useful.

Therefore, the present invention provides a method for correcting a genetic disease in a cell. The method includes the steps of providing a cell whose DNA includes a disease-causing mutated DNA sequence; exposing the cell to at least one gRNA that is complementary to a target site adjacent to the disease-causing mutated DNA sequence; exposing the cell to a Cpf1 endonuclease; directing the Cpf1 endonuclease to the target site with the at least one gRNA; causing a double stranded break in the DNA adjacent to the disease-causing mutated DNA sequence, with the Cpf1 endonuclease; exposing the cell to an isolated single stranded donor oligonucleotide including a wild type DNA sequence corresponding to the disease-causing mutated DNA sequence; replacing the disease-causing mutated DNA sequence with the wild type DNA sequence; and correcting the genetic disease.

With suitably designed gRNAs, the CRISPR/Cdf1 system will be effective at correcting genetic diseases, especially diseases caused by a single mutation, including, but not limited to, cystic fibrosis, severe combined immune deficiency, adenosine deaminase deficiency, chronic granulomatous disorder, hemophilia, Gaucher's Disease, and Rett Syndrome

CRISPR/Cpf1 Compositions and Methods for Genomic Sequencing and Diagnosis.

Recent advances in fluorescence imaging and image analysis have led to faster, simpler, more accessible techniques for diagnosis of disease and genetic analysis, including whole genome analysis. Through fluorescence labelling of specific DNA sequences or motifs, it is possible to visualize and quantitate integrated HIV-1 and other integrated viruses; measure the length of telomeres in genomic DNA, which are recognizable by repetitive TTAGGG sequences; count the copy number of genes and nucleotide repeats, such as the characteristic repeats of PolyQ disease; localize and quantitate retrotransposons associated with DNA damage and cancer risk; and sequence entire chromosomes with an instrument such as the BIONANO IRYS® system, which linearizes whole chromosomes and sequences them according to the positions of labelled DNA motifs.

The CRISPR/Cas9 system has been successfully modified to label, rather than cut, specific target sequences on a DNA strand. One strategy involves the use of the previously described catalytically deficient Cas9, and at least one gRNA to bind the catalytically deficient Cas9 to a specific target DNA sequence. The catalytically deficient Cas9 is labelled with a fluorescent polypeptide, or other detectable signal, so that its binding to the target sequence tags that sequence for detection. Alternatively, or in addition, one of more of the gRNAs can also be labelled, by means of an aptamer which is appended to one or more loops of the gRNA. The aptamer can bind to a dimerized bacteriophage coat protein, MS2, which is in turn can be fused with one single or multiple fluorescent proteins, such as EGFP.

Another strategy involves the use of one of the previously described nickase forms of Cas9. The nickase is directed to a target sequence by a suitable gRNA, to produce a nick in a single strand of DNA. Fluorophore-labelled nucleotides are provided at the site, as well as DNA polymerase. The nick is repaired with the fluorophore labelled nucleotides, creating a detectable label at the target sequence.

As previously stated, it is likely that catalytically deficient and nickase mutants of Cpf1 can be generated, using strategies similar to those used for Cas9. The CRISPR/Cpf1 system will therefore greatly expand the possibilities of genomic labelling, since it recognizes a set of target sequences that has very little overlap with those recognized by CRISPR/Cas9.

Therefore, the present invention provides a method for nick labelling a DNA sequence at a target site in a genome. The method includes the steps of exposing a DNA genome to a nickase mutant of a Cpf1 endonuclease; exposing the DNA genome to at least one guide gRNA that is complementary to a target sequence situated within the target site; directing the nickase mutant Cpf1 to the target sequence with the at least one gRNA; nicking the target sequence; creating a nicked target sequence; exposing the nicked target sequence to at least one labelled nucleotide (NT); incorporating the labelled NT into the nicked target sequence; and labelling the DNA sequence at the target site in the genome.

The present invention also includes a composition for labelling a DNA sequence at a target site in a genome. The composition includes at least one catalytically deficient Cpf1 endonuclease, and at least one guide RNA (gRNA), with the at least one gRNA being complementary to a target DNA sequence at the target site. A detectable label, such as a fluorescent label, is incorporated into the at least one catalytically deficient Cpf1 endonuclease, the at least one gRNA, or both.

Kits

The present invention also includes a kit to facilitate the application of the previously stated methods of treatment and prophylaxis of HIV infection. The kit includes a measured amount of a composition including at least one isolated nucleic acid sequence encoding an endonuclease, and at least one nucleic acid sequence encoding one or more gRNAs, wherein each of the gRNAs includes a spacer sequence complementary to a target sequence of an HIV provirus. The kit also includes and one or more items selected from the group consisting of packaging material, a package insert comprising instructions for use, a sterile fluid, a syringe and a sterile container. In a preferred embodiment, the nucleic acid sequences are included in an expression vector. The kit can also include a suitable stabilizer, a carrier molecule, a flavoring, or the like, as appropriate for the intended use.

In other embodiments, the kit further comprises one or more anti-viral agents and/or therapeutic reagents that alleviate some of the symptoms or secondary bacterial infections that may be associated with a flavivirus infection. Accordingly, packaged products (e.g., sterile containers containing one or more of the compositions described herein and packaged for storage, shipment, or sale at concentrated or ready-to-use concentrations) and kits, including at least one composition of the invention, e.g., a nucleic acid sequence encoding an endonuclease, for example, a Cpf1 endonuclease, and a guide RNA complementary to a target sequence in a retrovirus, or a vector encoding that nucleic acid and instructions for use, are also within the scope of the invention. A product can include a container (e.g., a vial, jar, bottle, bag, or the like) containing one or more compositions of the invention. In addition, an article of manufacture further may include, for example, packaging materials, instructions for use, syringes, delivery devices, buffers or other control reagents for treating or monitoring the condition for which prophylaxis or treatment is required.

The product may also include a legend (e.g., a printed label or insert or other medium describing the product's use (e.g., an audio- or videotape)). The legend can be associated with the container (e.g., affixed to the container) and can describe the manner in which the compositions therein should be administered (e.g., the frequency and route of administration), indications therefor, and other uses. The compositions can be ready for administration (e.g., present in dose-appropriate units), and may include one or more additional pharmaceutically acceptable adjuvants, carriers or other diluents and/or an additional therapeutic agent. Alternatively, the compositions can be provided in a concentrated form with a diluent and instructions for dilution.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

All documents mentioned herein are incorporated herein by reference. All publications and patent documents cited in this application are incorporated by reference for all purposes to the same extent as if each individual publication or patent document were so individually denoted. By their citation of various references in this document, applicants do not admit any particular reference is “prior art” to their invention.

### EXAMPLES

The present invention is further illustrated by the following specific examples. The examples are provided for illustration only and are not to be construed as limiting the scope or content of the invention in any way.

### Example 1: CRISPR/Cpf1 System for Inactivation and Elimination of Latent HIV-1

It has been determined that latent HIV-1 integrated into human T cells and other host cells can be inactivated and in many cases completely eradicated from the host genome, by a Cas9/gRNA system. Particularly effective were gRNAs complimentary to target sequences in the long terminal repeats (LTRs) of proviral HIV-1, especially target sequences in the U3 region. In some embodiments, pairs of gRNAs, with each member of a pair specific for a different target sequence, were especially effective in bringing about the excision of the entire stretch of DNA extending between the 5′ and 3′ LTRs (Hu W. et al., Proc Natl Acad Sci USA 111, 11461-11466 (2014); Khalili et al., 2015, International Patent Application No. WO2015/031775 to Khalili, et al.).

As previously stated, it is also known that Cpf1 endonucleases from Acidaminococcus and Lachnospiraceae are guided by gRNAs complementary to target sequences that extend approximately 24 nucleotides 3′ from the consensus PAM 5′-TTN (Zetsche B. et al., Cell 163, 1-13 Oct. 22, 2015). It is therefore likely that such nucleotide sequences in the HIV-1 LTRs will serve as target sequences for a Cpf/gRNA system. That is, gRNAs complimentary to at least a subset of the target sequences in the human HIV-1 LTR will cause inactivation and/or elimination of latent proviral HIV-1 when complexed with a Cpf1. TABLE 1 lists a set of target sequences defined in the human HIV-1 LTR. The target sequences are derived from the HIV-LTR nucleotide sequence as disclosed in FIG. 18 of International Patent Application No. WO2015/031775 to Khalili, et al. The sequences are classified according to the LTR region wherein the PAM (shown in parentheses) of each sequence is situated.

TABLE 1
Cpf1/gRNA TARGET SEQUENCES IN THE
HUMAN HIV-1 LTR.
U3 Region:
(TTA) CACCCTGTGAGCCTGCATGGGATG (SEQ ID NO: 1);
(TTA) GAGTGGAGGTTTGACAGCCGCCTA (SEQ ID NO: 2);
(TTT) GGATGGTGCTACAAGCTAGTACCA (SEQ ID NO: 3);
(TTT) GACAGCCGCCTAGCATTTCATCAC (SEQ ID NO: 4);
(TTT) CATCACATGGCCCGAGAGCTGCAT (SEQ ID NO: 5);
(TTT) CCGCTGGGGACTTTCCAGGGAGGC (SEQ ID NO: 6);
(TTT) CCAGGGAGGCGTGGCCTGGGCGGG (SEQ ID NO: 7);
(TTT) TTGCTTGTACTGGGTCTCTCTGGT (SEQ ID NO: 8);
(TTT) TGCTTGTACTGGGTCTCTCTGGTT (SEQ ID NO: 9);
(TTT) GCTTGTACTGGGTCTCTCTGGTTA (SEQ ID NO: 10);
(TTC) ACTCCCAACGAAGACAAGATATCC (SEQ ID NO: 11);
(TTC) CCTGATTGGCAGAACTACACACCA (SEQ ID NO: 12);
(TTC) ATCACATGGCCCGAGAGCTGCATC (SEQ ID NO: 13);
(TTC) AAGAACTGCTGACATCGAGCTTGC (SEQ ID NO: 14);
(TTC) CGCTGGGGACTTTCCAGGGAGGCG (SEQ ID NO: 15);
(TTC) CAGGGAGGCGTGGCCTGGGCGGGA (SEQ ID NO: 16);
(TTG) ATCTGTGGATCTACCACACACAAG (SEQ ID NO: 17);
(TTG) GCAGAACTACACACCAGGGCCAGG (SEQ ID NO: 18);
(TTG) GATGGTGCTACAAGCTAGTACCAG (SEQ ID NO: 19);
(TTG) AGCAAGAGAAGGTAGAAGAAGCCA (SEQ ID NO: 20);
(TTG) TTACACCCTGTGAGCCTGCATGGG (SEQ ID NO: 21);
(TTG) CTACAAGGGACTTTCCGCTGGGGA (SEQ ID NO: 22);
(TTG) CTTGTACTGGGTCTCTCTGGTTAG (SEQ ID NO: 23);
(TGG) TACTGGGTCTCTCTGGTTAGACCA (SEQ ID NO: 24);
R Region
(TTA) GACCAGATCTGAGCCTGGGAGCTC (SEQ ID NO: 25);
(TTA) AGCCTCAATAAAGCTTGCCTTGAG (SEQ ID NO: 26);
(TTG) CCTTGAGTGCTTCAAGTAGTGTGT (SEQ ID NO: 27);
(TTG) AGTGCTTCAAGTAGTGTGTGCCCG (SEQ ID NO: 28);
U5 Region
(TTA) GTCAGTGTGGAAAATCTCTAGCA (SEQ ID NO: 29);
(TTC) AAGTAGTGTGTGCCCGTCTGTTGT (SEQ ID NO: 30);
(TTT) TAGTCAGTGTGGAAAATCTCTAGC (SEQ ID NO: 31);
(TTT) AGTCAGTGTGGAAAATCTCTAGCA (SEQ ID NO: 32);
(TTG) AGTGCTTCAAGTAGTGTGTGCCCG (SEQ ID NO: 33).

The present invention includes a gRNA complementary to each of target sequences listed in TABLE 1. A gRNA of the present invention may or may not include a sequence complementary to the PAM sequence of a target sequence. A gRNA may be complementary to a truncated variation of a listed sequence, for example one that is truncated by 1, 2, 3, or more nucleotides on the 3′ end. A gRNA may be less than 100% complementary a target sequences listed in TABLE 1. For example, a gRNA can be 75% complementary to a listed target sequence, or 80% complementary to a listed target sequence, 85%, or 90%, or 95%, 96%, 97%, 98%, 99% complementary to a listed target sequence. The present invention includes gRNAs that are complementary to the antisense strand of each of the listed target sequences, or 95% complementary, or complementary to an antisense sequence that is truncated by 1, 2, 3, or more nucleotides. It will be understood that Table 1 includes only a representative sample of target sequences in the HIV-1 LTRs. Additional sequences adjacent to different PAMS may also exist, and also within the scope of the present invention.

In certain embodiments, a composition for inactivating a target gene in the genome of a host cell in vitro or in vivo, comprises at least one isolated nucleic acid sequence encoding a Cpf1 (CRISPR from Prevotella and Francisella 1) endonuclease, and at least one guide RNA (gRNA), said at least one gRNA having a complementary sequence identity of at least 75% to a target sequence in the target gene. In certain embodiments, the at least one gRNA comprises a complementary sequence identity of at least 95% to a target sequence in the target gene. In other embodiments, the at least one gRNA is complementary to a target sequence in the target gene. In certain embodiments, a target gene comprises coding and non-coding nucleic acid sequences of a retroviral genome, for example, a human immunodeficiency virus (HIV). In certain embodiments, the non-coding region comprises a long terminal repeat of HIV or a sequence within the long terminal repeat of HIV. In other embodiments, the sequence within the long terminal repeat of HIV comprises a sequence within U3, R, or U5 regions.

The gRNAs are in certain embodiments in a multiplex configuration, either encoded by the same vector or physically separate vectors. Each vector can encode single gRNAs or a plurality of gRNA having a combination of complementary sequence identities to one or more target sequences. Accordingly, in certain embodiments, the composition comprises a plurality of guide RNA nucleic acid sequences complementary to a plurality of target nucleic acid sequences of human immunodeficiency virus.

In some embodiments, a target gene comprises at least a 75% sequence identity to any one of sequences comprising SEQ ID NOS: 1 to 33. In other embodiments, a target gene comprises any one of sequences comprising SEQ ID NOS: 1 to 33.

As discussed above, in certain embodiments, one isolated nucleic acid sequence encoding a Cpf1 (CRISPR from Prevotella and Francisella 1) endonuclease, and an isolated nucleic acid sequence encoding the at least one guide RNA (gRNA), are expressed by the same vector. In other embodiments, one isolated nucleic acid sequence encoding a Cpf1 (CRISPR from Prevotella and Francisella 1) endonuclease, is expressed by a first vector and an isolated nucleic acid sequence encoding said at least one guide RNA (gRNA) is expressed by a second vector.

In other embodiments, the composition optionally comprises one or more: anti-viral agents, chemotherapeutic agents, anti-fungal agents, anti-parasitic agents, anti-bacterial agents, anti-inflammatory agents immunomodulating agents or combinations thereof. In other embodiments, any one or more of these agents can be combined in a co-therapeutic treatment by administering to a subject in need thereof, one or more of these agents at the same time as the compositions embodied herein, or before administration of the compositions embodied herein, after administration of the compositions embodied herein or as part of a normal therapeutic strategy.

The gRNAs of the present invention are synthesized generally as described by Zetsche B. et al., Cell 163, 1-13 Oct. 22, 2015. Cloning of the gRNAs into vectors for expression in host cells is as described in Hu, et al., 2014, and in WO2015/031775 to Khalili, et al., both of which are incorporated in their entirety. Screening of Cpf1/gRNA combinations for gene editing activity is performed by genomic analyses, Surveyor assays, and assays of viral infection, activation, and expression, as disclosed in Hu W. et al., Proc Natl Acad Sci USA 111, 11461-11466 (2014), and in WO2015/031775 to Khalili, et al.

The invention has been described in an illustrative manner, and it is to be understood that the terminology that has been used is intended to be in the nature of words of description rather than of limitation. Obviously, many modifications and variations of the present invention are possible in light of the above teachings. It is, therefore, to be understood that within the scope of the appended claims, the invention can be practiced otherwise than as specifically described.

What is claimed is:

1. A composition for use in inactivating a target gene in the genome of a host cell in vitro or in vivo, comprising:
at least one isolated nucleic acid sequence encoding a Cpf1 (CRISPR from Prevotella and Francisella 1) endonuclease, and
at least one guide RNA (gRNA), said at least one gRNA having a complementary sequence identity of at least 75% to a target sequence in the target gene.

2. The composition of claim 1, wherein said at least one gRNA comprises a complementary sequence identity of at least 95% to a target sequence in the target gene.

3. The composition of claim 2, wherein said at least one gRNA is complementary to a target sequence in the target gene.

4. The composition of claim 1, wherein a target gene comprises coding and non-coding nucleic acid sequences of a retroviral genome.

5. The composition of claim 4, wherein the retrovirus is human immunodeficiency virus (HIV).

6. The composition of claim 4, wherein the non-coding region comprises a long terminal repeat of HIV or a sequence within the long terminal repeat of HIV.

7. The composition of claim 6, wherein the sequence within the long terminal repeat of HIV comprises a sequence within U3, R, or U5 regions.

8. The composition of claim 1, further comprising a plurality of guide RNA nucleic acid sequences complementary to a plurality of target nucleic acid sequences of human immunodeficiency virus.

9. The composition of claim 1, wherein a target gene comprises at least a 75% sequence identity to any one of sequences comprising SEQ ID NOS: 1 to 33.

10. The composition of claim 1, wherein a target gene comprises any one of sequences comprising SEQ ID NOS: 1 to 33.

11. The composition of claim 1, wherein the one isolated nucleic acid sequence encoding a Cpf1 (CRISPR from Prevotella and Francisella 1) endonuclease, and an isolated nucleic acid sequence encoding said at least one guide RNA (gRNA) are expressed by a vector.

12. The composition of claim 1, wherein the one isolated nucleic acid sequence encoding a Cpf1 (CRISPR from Prevotella and Francisella 1) endonuclease, is expressed by a first vector and an isolated nucleic acid sequence encoding said at least one guide RNA (gRNA) is expressed by a second vector.

13. The composition of claim 1, optionally comprising one or more: anti-viral agents, chemotherapeutic agents, anti-fungal agents, anti-parasitic agents, anti-bacterial agents, anti-inflammatory agents immunomodulating agents or combinations thereof.

14. (canceled)

15. (canceled)

16. A composition for use in inactivating an integrated proviral DNA in the genome of a host cell, including:
at least one isolated nucleic acid sequence encoding a Cpf1 (CRISPR from Prevotella and Francisella 1) endonuclease, and
at least one guide RNA (gRNA), said at least one gRNA being complementary to a target sequence in a proviral DNA.

17. The composition according to claim 16, wherein the proviral DNA comprises a proviral DNA of human immunodeficiency virus-1 (HIV-1), human immunodeficiency virus-2 (HIV-2), human T cell lymphotropic virus type I (HTLV-1), human T cell lymphotropic virus type II (HTLV-II), herpes simplex virus type 1 (HSV-1), herpes simplex virus type 2 (HSV-2), or JC virus (JCV).

18. The composition according to claim 17, wherein the proviral DNA is an HIV-1 DNA, and said at least one gRNA is complementary to a target sequence in the HIV-1 DNA.

19. The composition according to claim 18, wherein said at least one gRNA is complementary to a target sequence in the long terminal repeat (LTR) of the HIV-1 DNA.

20. A method of inactivating an integrated proviral DNA in the genome of a host cell, including the steps of:
treating a host cell having an integrated proviral DNA with at least one isolated nucleic acid sequence encoding a Cpf1 (CRISPR from Prevotella and Francisella 1) endonuclease;
treating the host cell with at least one isolated nucleic acid sequence encoding at least one guide RNA (gRNA), the at least one gRNA being complementary to a target sequence in the proviral DNA; and
inactivating the proviral DNA.

21. The method according to claim 20, wherein the proviral DNA comprises a proviral DNA of human immunodeficiency virus-1 (HIV-1), human immunodeficiency virus-2 (HIV-2), human T cell lymphotropic virus type I (HTLV-I), human T cell lymphotropic virus type II (HTLV-II), herpes simplex virus type 1 (HSV-1), herpes simplex virus type 2 (HSV-2), or JC virus (JCV).

22. The method according to claim 21, wherein the target sequence is situated in a proviral HIV-1 DNA.

23. The method according to claim 22, wherein the target sequence situated in the HIV-1 proviral DNA is a target sequence situated in a long terminal repeat (LTR) of the proviral HIV-1 DNA.

24. A vector composition for use in inactivating a target gene the genome of a host cell in vitro or in vivo, comprising:
at least one isolated nucleic acid sequence encoding a Cpf1 (CRISPR from Prevotella and Francisella 1) endonuclease, and
at least one guide RNA (gRNA), said at least one gRNA being complementary to a target sequence in the target gene,
said at least one isolated nucleic acid sequence encoding said at least one Cpf1 endonuclease, and said at least one gRNA, being included in at least one expression vector,
wherein said at least one expression vector induces the expression of said at least one Cpf1 endonuclease, and said at least one gRNA, in a host cell.

25. The vector composition according to claim 24, wherein said at least one expression vector includes a lentiviral expression vector.

26. (canceled)

27. (canceled)

28. A pharmaceutical composition for the inactivation of an integrated provirus in the cells of a mammalian subject, comprising:
an isolated nucleic acid sequence encoding a Cpf1 (CRISPR from Prevotella and Francisella 1) endonuclease; and
at least one isolated nucleic acid sequence encoding at least one guide RNA (gRNA) that is complementary to a target sequence in a proviral DNA;
said isolated nucleic acid sequences being included in at least one expression vector.

29. The pharmaceutical composition according to claim 28, wherein the provirus comprises: human immunodeficiency virus-1 (HIV-1), human immunodeficiency virus-2 (HIV-2), human T cell lymphotropic virus type I (HTLV-I), human T cell lymphotropic virus type II (HTLV-II), herpes simplex virus type 1 (HSV-1), herpes simplex virus type 2 (HSV-2), or JC virus (JCV).

30. The pharmaceutical composition according to claim 28, further comprising one or more: anti-viral agents, chemotherapeutic agents, anti-fungal agents, anti-parasitic agents, anti-bacterial agents, anti-inflammatory agents immunomodulating agents or combinations thereof.

31. (canceled)

32. (canceled)

33. (canceled)

34. (canceled)

35. (canceled)

36. (canceled)

37. (canceled)

38. (canceled)

39. (canceled)

40. (canceled)
